SEQUENCE LISTING (14 sequences)

SEQ ID NO: 1 - DNA, 21 bp, Artificial Sequence
  Description of Artificial Sequence Synthetic polynucleotide sequence
aattgtgagcggataacaatt

SEQ ID NO: 2 - DNA, 65 bp, Artificial Sequence
  Description of Artificial Sequence Synthetic polynucleotide sequence
ctgtgtagatagtttactgccctgaaccctgatcgaaagacctggtcacaaattgttatccgctc

SEQ ID NO: 3 - DNA, 69 bp, Artificial Sequence
  Description of Artificial Sequence Synthetic polynucleotide sequence
acacagcagactgagtttgatccattaacgaaccttcacgatacctgacgacttgtggaattgtgagcg

SEQ ID NO: 4 - DNA, 74 bp, Artificial Sequence
  Description of Artificial Sequence Synthetic polynucleotide sequence
acacagcagactgagtttgatccattaacgaaccttcacgatacctgacgacttgtggaattgtgagcggataa

SEQ ID NO: 5 - DNA, 50 bp, Artificial Sequence
  Description of Artificial Sequence Synthetic polynucleotide sequence
ggggacaagtttgtacaaaaaagcaggcaccatggaggagccgcagtcag

SEQ ID NO: 6 - DNA, 54 bp, Artificial Sequence
  Description of Artificial Sequence Synthetic polynucleotide sequence
ggggaccactttgtacaagaaagctgggttgcggtctgagtcaggcccttctgt

SEQ ID NO: 7 - DNA, 49 bp, Artificial Sequence
  Description of Artificial Sequence Synthetic polynucleotide sequence
ggggacaagtttgtacaaaaaagcaggcaccatgtgcaataccaacatg

SEQ ID NO: 8 - DNA, 49 bp, Artificial Sequence
  Description of Artificial Sequence Synthetic polynucleotide sequence
ggggaccactttgtacaagaaagctgggttgcgggggaaataagttagc

SEQ ID NO: 9 - DNA, 20 bp, Artificial Sequence
  Description of Artificial Sequence Synthetic polynucleotide sequence
ccctgaaccctgatcgaaag

SEQ ID NO: 10 - DNA, 24 bp, Artificial Sequence
  Description of Artificial Sequence Synthetic polynucleotide sequence
ttaacgaaccttcacgatacctga

SEQ ID NO: 11 - DNA, 31 bp, Artificial Sequence
  Description of Artificial Sequence Synthetic polynucleotide sequence
tggtcacaaattgttatccgctcacaattcc

SEQ ID NO: 12 - DNA, 69 bp, Artificial Sequence
  Description of Artificial Sequence Synthetic polynucleotide sequence
acgtatgctatgctatgagtatgaacacagaagactgagttagatcgacgacttgtggaattgtgagcg

SEQ ID NO: 13 - DNA, 31 bp, Artificial Sequence
  Description of Artificial Sequence Synthetic polynucleotide sequence
tatgagtatgaacacagaagactgagttaga

SEQ ID NO: 14 - DNA, 31 bp, Artificial Sequence; identical to SEQ ID NO: 11
  Description of Artificial Sequence Synthetic polynucleotide sequence

(19) United States
(12) Patent Application Publication
(10) Pub. No.: US 2005/0095627 A1
(43) Pub. Date: May 5, 2005

(54) Multiple antigen detection assays and reagents

(75) Inventors: John L. Kolman, Escondido, CA (US); Geoffrey M. Wahl, San Diego, CA (US)

Correspondence Address: BIOTECHNOLOGY LAW GROUP, 658 MARSOLAN AVENUE, SOLANA BEACH, CA, 92075, US

(73) Assignee: THE SALK INSTITUTE FOR BIOLOGICAL STUDIES, La Jolla, CA (US)

(21) Appl. No.: 10/933,671

(22) Filed: Sep. 3, 2004

Related U.S. Application Data
(60) Provisional application No. 60/500,114, filed on Sep. 3, 2003.

(51) Int. Cl. C12Q 1/68

(52) U.S. Cl. 435/6

(57) ABSTRACT

Provided herein are multiple antigen detection (MAD) assays and reagents for performing such assays. MAD assay embodiments are useful for identifying proximal antigens in a sample (e.g., antigens existing in a complex), and are performed by contacting a sample with two or more hybrids, where each hybrid comprises a nucleic acid and a binding partner that specifically binds to an antigen. In these embodiments, the hybrid nucleic acids hybridize to one another when the hybrids bind to proximal antigens, and the hybridization product then is detected, often by a polymerase chain reaction (PCR) process or a nucleic acid binding agent comprising a detectable label. MAD assays and reagents are applicable to antigen detection in situ, chromatin immunoprecipitation and genomic sequencing, detection of proximal intramolecular regions in an antigen, detecting antigen multimerization, diagnostics and therapeutics. Also provided are kits for conducting MAD applications.

### RELATED APPLICATION

This application claims the benefit of Patent Application No. 60/500,114, filed on Sep. 3, 2003, having attorney docket nos. 532793000200 and SALK-2018-PRVI, entitled “Multiple Antigen Detection Assays and Reagents,” and naming John L. Kolman and Geoffrey M. Wahl as inventors. This related application hereby is incorporated herein by reference in its entirety, including all text, tables and drawings.

### STATEMENT OF GOVERNMENT SUPPORT

This invention was made in part with government support under Grant Nos. R01CA6 1449 and R01CA48405 awarded by the National Institutes of Health. The government has certain rights in this invention.

### FIELD OF THE INVENTION

The invention concerns methods for sensitively detecting proximal molecules and binding regions in a biological sample.

### BACKGROUND

A process known as the polymerase chain reaction (PCR) amplifies a nucleic acid sequence by several orders of magnitude and has been utilized to detect nucleic acid antigens in a sample. PCR involves repeated cycles of DNA polymerase primer extension reactions. In each cycle, the target DNA is heat denatured in the presence of two oligonucleotides, where each oligonucleotide includes a sequence complementary to a subsequence in the nucleic acid that flanks the target sequence. Each oligonucleotide hybridizes to the nucleic acid on each side of the target sequence and functions as a primer for an extension reaction catalyzed by DNA polymerase. In the extension reaction, the target DNA sequence is copied by DNA polymerase yielding a copy of each strand of the targeted sequence. By repeating each cycle of heat denaturation, primer hybridization and primer extension, the target nucleic acid sequence is amplified by orders of magnitude in a short period of time. For example, DNA sometimes is amplified a million fold or more in approximately two to four hours.

PCR also has been applied to assays for detecting protein antigens in biological samples. In these applications, a DNA molecule sometimes is conjugated to an antibody that binds to the protein antigen or to an antibody that binds the assayed protein. The DNA molecule often is double stranded and is linked indirectly to the antibody. For example, double-stranded DNA has been biotinylated and linked to an avidin-derivitized antibody; biotinylated DNA has been linked to a biotinylated antibody via avidin; and biotinylated DNA has been indirectly joined to an antibody by a protein A-streptavidin chimera. Single-stranded DNA also has been linked to an antibody by a chemical cross linker. Antibody-DNA conjugates have been utilized in enzyme-linked immunosorbant assays (ELISAs), where the protein antigen is immobilized to a solid support by a non-conjugate antibody or by other means, the immobilized antigen is contacted with the antibody-DNA conjugate and washed to remove unbound conjugate, and the DNA of the bound conjugate then is detected by PCR. The conjugate antibody often binds to the Fc region of an antibody already bound to the immobilized protein antigen in a sandwich format to report the presence of the protein antigen.

### SUMMARY

An organism contains many different cell types, and each cell comprises many distinct molecules, including lipids, nucleic acids, and proteins. Subgroups of molecules can aggregate in different combinations and form complexes. Complexes having molecules such as proteins regulate signaling processes in cells, and these signaling processes control cellular events such as DNA replication, DNA transcription, polypeptide synthesis, cell growth, cell differentiation, cell proliferation and cell death, for example. These cellular events are relevant to disease progression, where disruptions in signaling processes that control cell proliferation and cell death, for example, lead to cancer.

Proteins in the complexes often are bound to other proteins and sometimes are bound to nucleic acids (e.g., DNA). Whether a molecule enters or leaves a complex often is determined by the presence or absence of a covalent modification on the molecule's surface (e.g., phosphorylation or ubiquination). The enzymes that add or remove covalent modifications to or from molecules often are members of a complex.

The presence or absence of one molecular member can lead to a functioning or non-functioning complex, and therefore, the molecular composition of the complex controls its biological function or alters the specificity or activity of the complex. Accordingly, reagents and methods for rapidly, specifically, and sensitively determining whether particular molecules are co-localized and proximal to one another in a cell are useful for determining which molecules are present in a complex. By determining whether specific molecules are proximal under varying cellular conditions, these reagents and methods are useful for elucidating complex-mediated signaling processes and biological events. Elucidating complex-mediated signaling processes and biological events that lead to a disease is useful for designing therapies that specifically target the disease.

Thus, reagents and methods for performing multiple antigen detection (MAD) assays have been developed for determining whether specific cellular molecules in a sample or binding sites in a molecule are in proximity to one another. MAD assays are highly sensitive because a detection process that detects low antigen concentrations is utilized, such as an amplification process (e.g., a polymerase chain reaction (PCR) process (a “MAD-PCR” embodiment)) or a labeled nucleic acid binding agent (a “MAD-TAG” embodiment), for example. In MAD assays, a biological sample is contacted with a reagent (hereafter referred to as a “hybrid”), which comprises (1) a molecule that specifically binds to an assayed molecule (referred to hereafter as a “binding partner”) and (2) a nucleic acid conjugated to the binding partner. MAD assays also are highly specific (e.g., MAD assays produce few false-positive results) because a signal selectively is generated when the assayed molecule or molecules are in proximity to one another. Reasons for this specificity are that nucleic acids on different hybrids hybridize to form a hybridization product when the hybrids and target binding sites are in proximity to one another and/or when there is a sufficient local concentration of the target binding sites.

Hence, provided are methods for determining whether a first molecule and a second molecule are in proximity in a sample. These methods also are referred to herein as methods for detecting a first molecule and a second molecule in a sample as the molecules are detected when they are in proximity to one another. The methods comprise contacting the sample with a first hybrid, which comprises a first binding partner and a first nucleic acid, and a second hybrid, which comprises a second binding partner and a second nucleic acid. The first binding partner specifically binds to the first molecule and the second binding partner specifically binds to the second molecule. The first nucleic acid comprises a first nucleotide sequence complementary to a second nucleotide sequence in the second nucleic acid and is capable of forming a hybridization product with the second nucleic acid when the first hybrid is bound to the first molecule, the second hybrid is bound to the second molecule, and the first molecule and the second molecule are in proximity. Hybridization products having non-overlapping regions often are extended to form an extension product. The presence or absence of the hybridization product or the extension product then is identified, where the presence of the hybridization product or extension product is indicative of the first molecule and the second molecule being in proximity in the sample, and the presence of a hybridization product or extension product detects the first molecule and the second molecule in the sample. These methods also are applicable to detecting one molecule in a sample, where the hybrid binding partners bind to binding regions in the molecule.

The sample and the hybrids sometimes are diluted in assay embodiments after the hybrids and sample are contacted, where the molecules and hybrids at this stage of the assay are not in association with a solid phase. An agent that inhibits non-specific interactions between molecules in the sample (e.g., a blocking agent) sometimes is included at the time of dilution, or before or after dilution in certain embodiments. The sample and hybrids sometimes are washed after a sample is contacted with one or more hybrids in heterogeneous assay embodiments where a molecule and/or hybrid is in association with a solid phase. Dilution and/or washing often are performed before the hybridization product is extended and before the hybridization product or extension product is detected.

Each assayed molecule comprises one or more binding sites to which one or more hybrid binding partners bind, where each binding site sometimes is referred to herein as a “binding region” or “antigen.” Each molecule often is a protein and sometimes is a non-hybrid nucleic acid, such as double-stranded DNA from a cell (hereafter referred to as “genomic DNA” or “chromatin DNA”). The binding partner often is an antibody (e.g., a monoclonal antibody) or antibody fragment and the hybrid nucleic acid frequently is single-stranded DNA (ssDNA). A hybridization product formed by hybrid nucleic acids often includes an overlapping region and a non-overlapping region (i.e., a double-stranded region and a single-stranded region), where the overlapping region sometimes is six or fewer nucleotides in length (e.g., 3 to 6 nucleotides, 4 to 6 nucleotides, 5 to 6 nucleotides, or 5 nucleotides in length). The hybridization product sometimes is detected by extending the ends of each nucleic acid, amplifying the extended product by PCR and detecting the amplification products.

The hybridization product or an extension product thereof sometimes is detected by a nucleic acid binding agent. Thus, provided is a method for detecting a first molecule and a second molecule in a sample, which comprises contacting a sample with a first hybrid and a second hybrid, where the first hybrid comprises a first binding partner and a first nucleic acid and the second hybrid comprises a second binding partner and a second nucleic acid. The first binding partner specifically binds to the first molecule and the second binding partner specifically binds to the second molecule. The first nucleic acid comprises a first nucleotide sequence complementary to a second nucleotide sequence in the second nucleic acid and is capable of forming a hybridization product with the second nucleic acid when the first hybrid is bound to the first molecule and the second hybrid is bound to the second molecule and the first molecule and the second molecule are in proximity. The hybridization product optionally is extended to form an extension product and the hybridization product or extension product is contacted with a nucleic acid binding agent that specifically binds to a nucleotide sequence in the hybridization product or extension product. The nucleic acid binding agent is added at any time during the assay, where the agent is added before or at the same time hybrids are added and the hybridization product or extension product are contacted by the agent when they are formed, or the agent is added to the assay after the hybridization product or extension product are formed, for example. The presence or absence of the nucleic acid binding agent bound to the hybridization product or extension product is identified, whereby identifying the nucleic acid binding agent bound to the hybridization product or extension product detects the first molecule and the second molecule in the sample. The molecules and hybrids sometimes are diluted and/or washed before the hybridization product is extended. These methods also are applicable for detecting one molecule in a sample, where each hybrid binding partner binds to a binding region in the molecule.

The nucleic acid binding agent often specifically binds to a nucleotide sequence of the hybridization product or extension product. The nucleic acid binding agent often is a protein, including but not limited to, a lac repressor, a gal4 or a tus protein, or a fragment of the foregoing and/or a sequence variant of the foregoing, which specifically binds to a nucleotide sequence in the hybridization product or extension product. The nucleic acid binding agent sometimes is a nucleic acid that hybridizes to the hybrid nucleic acids, and sometimes forms a triplex structure with extended hybrid complexes. The nucleic acid binding agent often comprises a detectable label, and in certain embodiments, it comprises a cytotoxic agent or a solid support. Nucleic acid binding agents are useful in diagnostic applications and therapeutic applications described in greater detail hereafter.

A signal enhancer nucleic acid sometimes is utilized in some embodiments. The signal enhancer nucleic acid specifically hybridizes to a single stranded portion of the first nucleic acid or second nucleic acid, and where the 5′ end of the signal enhancer nucleic acid abuts the 3′ end of the other of the first nucleic acid or the second nucleic acid. The term “abuts” often refers to a configuration in which there is no gap between the 5′ end of the signal enhancer nucleic acid and the 3′ end of the first or second nucleic acid, and sometimes refers to a configuration in which there is a gap of one or two nucleotides between the 5′ end of the signal enhancer nucleic acid and the 3′ end of the first or second nucleic acid. The signal enhancer nucleic acid often enhances detection of an extension product in MAD assays, and sometimes is referred to herein as a “signal enhancer nucleic acid.” Without being bound by theory, it is expected that the signal enhancer nucleic acid facilitates binding of a polymerase used to extend the hybridization product. The signal enhancer nucleic acid is added at any stage of a MAD assay, including at the same time the sample is contacted with hybrids, after molecules and hybrids are diluted or washed and before a hybridization product is extended, for example.

Also provided is a method for determining whether a first molecule and a second molecule are in proximity in a cell, which comprises contacting the cell with a fust hybrid, which comprises a first binding partner and a first nucleic acid, and a second hybrid, which comprises a second binding partner and a second nucleic acid. As in methods described above, the first binding partner specifically binds to the first molecule and the second binding partner specifically binds to the second molecule. The first nucleic acid comprises a first nucleotide sequence complementary to a second nucleotide sequence in the second nucleic acid and is capable of forming a hybridization product with the second nucleic acid when the first hybrid is bound to the first molecule and the second hybrid is bound to the second molecule and the first molecule and the second molecule are in proximity. The hybridization product optionally is extended to form an extension product and the presence or absence of the hybridization product or the extension product is detected, whereby the presence of the hybridization product or extension product is indicative of the first molecule and the second molecule being in proximity in the cell. Cells sometimes are fixed to a solid support. Other embodiments described herein are applicable, such as washing fixed cells after they are contacted with hybrids and detecting the extension product by PCR or a nucleic acid binding agent, for example.

Provided also is a method for detecting two or more binding regions in a target molecule, which comprises contacting the target molecule with a first hybrid, which comprises a first binding partner and a first nucleic acid, and a second hybrid, which comprises a second binding partner and a second nucleic acid. The first binding partner specifically binds to a first binding region of the target molecule and the second binding partner specifically binds to a second binding region of the target molecule. The first nucleic acid comprises a first nucleotide sequence complementary to a second nucleotide sequence in the second nucleic acid and is capable of forming a hybridization product with the second nucleic acid when the first hybrid is bound to the first binding region and the second hybrid is bound to the second binding region and the first binding region and the second binding region are in the target molecule. Hybridization products having a non-overlapping region optionally are extended to form extension products; and the presence or absence of the hybridization products or the extension products are detected, whereby the presence of the hybridization products or extension products is indicative of the first binding region and the second binding region being in the target molecule. Other embodiments described herein are applicable, such as diluting or washing after the sample is contacted by hybrids, and detecting the extension product by PCR or a nucleic acid binding agent, for example.

Also provided is a method for identifying a multimer of a target molecule in a sample, which comprises contacting the sample with a first hybrid, which comprises a first binding partner and a first nucleic acid, and a second hybrid, which comprises a second binding partner and a second nucleic acid. The first binding partner is identical to the second binding partner and specifically binds to the target molecule. The first nucleic acid comprises a first nucleotide sequence complementary to a second nucleotide sequence in the second nucleic acid and is capable of forming a hybridization product with the second nucleic acid when the first hybrid and the second hybrid are bound to a multimer of the target molecule. Hybridization products having non-overlapping regions optionally are extended to form extension products, and the presence or absence of the hybridization products or extension products are detected, whereby the presence of the hybridization products or extension products is indicative of a multimer of the target molecule. Other embodiments described herein are applicable, such as diluting or washing after the sample is contacted by hybrids, and detecting the extension product by PCR or a nucleic acid binding agent, for example.

Provided also are compositions and kits for assembling MAD assay reagents and conducting MAD assays, diagnostics and therapeutic methods. For example, provided is a composition or kit which comprises a nucleic acid derivitized at the 5′ end with a sulfhydryl- or amine-reactive chemical moiety (e.g., a maleimide or haloacetyl sulfhydryl-reactive moiety or a isotriocyanate, succinyl ester or sulfonyl halide amine-reactive moiety) and a reagent for coupling the nucleic acid derivative to a binding partner (e.g., a reducing agent such as dithiothreitol (DTT) for coupling a haloacetyl- or maleimide-derivitized nucleic acid to an antibody or antibody fragment). Also provided is a composition or kit which comprises one or more hybrids capable of forming a hybridization product having an overlapping, partially double stranded region of six or fewer nucleotides in length. Provided also is a composition or kit which comprises one or more hybrids in combination with a nucleic acid binding agent. Also provided is a composition or kit which comprises one or more hybrids in combination with a signal enhancer nucleic acid described herein. Provided also is a composition or kit which comprises one or more hybrids, where a binding partner of one or more of the hybrids often is an antibody or antibody fragment, and the antibody or antibody fragment specifically binds to a particular antibody isotype (e.g., IgG₁ or IgG₂) or an antibody from a specific animal (e.g., a mouse or rat monoclonal antibody or a mouse, rat, rabbit, goat, hamster or chicken polyclonal antibody).

These and other MAD assays and reagents are described further hereafter in the detailed description, claims, and drawings.

### BRIEF DESCRIPTION OF DRAWINGS

FIG. 1 illustrates a MAD assay embodiment in which reagents selectively and sensitively generate a signal when the molecules detected are in proximity to one another. FIG. 1 shows hybrid nucleic acids hybridize when molecules P1 and P2 are in proximity to one another, but do not detectably hybridize when the molecules are not in proximity. In this embodiment, the hybridization product formed by the hybrid nucleic acids is extended and the extension product is detected by a polymerase chain reaction (PCR) process.

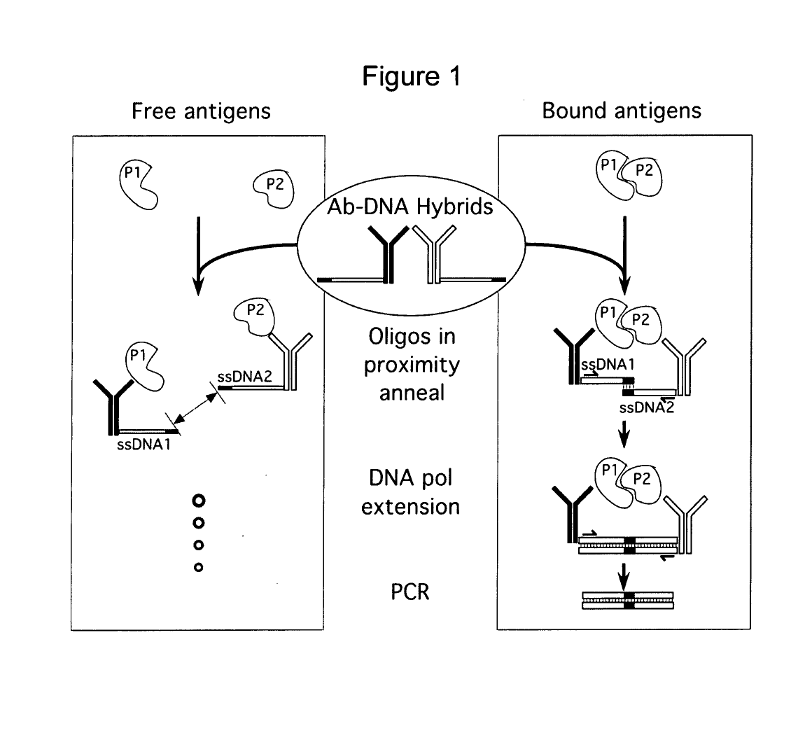

FIG. 2 illustrates a MAD assay embodiment designated “MAD-TAG” in which an extension product is identified by a detectably labeled nucleic acid binding agent that specifically binds to the nucleotide sequence of the extension product.

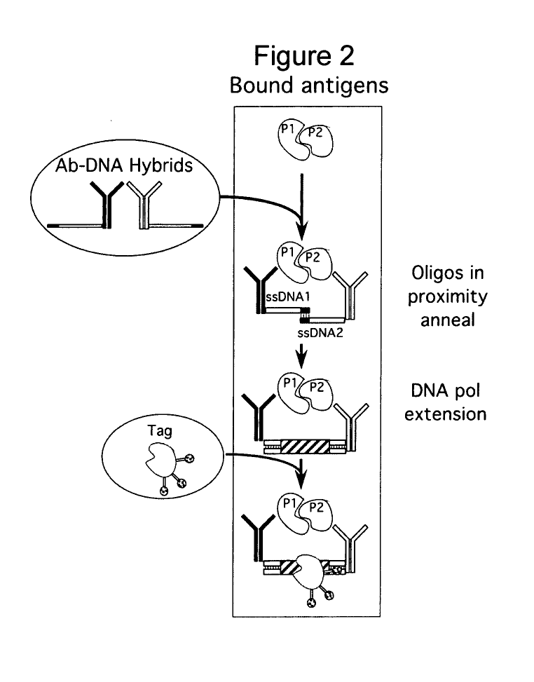

### DETAILED DESCRIPTION

Several MAD assays and reagents are provided herein, including but not limited to assays and reagents for detecting proximal molecules in a sample; detecting a molecule having proximal binding sites in a sample or detecting the presence of proximal regions in a molecule; detecting proximal molecules in situ (e.g., in a cell); determining the presence of proximal molecules linked directly or indirectly to chromatin DNA (hereafter referred to as “chromatin immunoprecipitation” and “MAD-ChIP”); determining a nucleotide sequence of a non-hybrid nucleic acid, where the non-hybrid nucleic acid is a proximal molecule or is linked directly or indirectly to a proximal molecule; detecting multimers of a molecule in a sample; and sensitively detecting several molecules in a sample (referred to hereafter as “multiplexing”). The methods sometimes are carried out by exposing samples to different specific conditions, as described in greater detail hereafter. MAD assays sometimes are carried out in a homogeneous format (e.g., assay components are not immobilized to a solid support) and sometimes are performed in a heterogeneous format (i.e., one or more assay components are in association with a solid phase). Many MAD embodiments are applicable to diagnostic applications, for example, methods of diagnosing a disease by assaying a sample from a subject, and some MAD embodiments are applicable to treatment applications, for example, methods of treating a disease in a subject by administering one or more MAD assay reagents to the subject.

MAD Hybrids

MAD reagents include hybrids that specifically bind to a binding site of a molecule in a sample. A “binding site,” which also is referred to as a “binding region” or “antigen” herein, typically is a portion of a molecule that directly contacts the hybrid when the hybrid is bound to the molecule. The term “specifically binds” refers to the hybrid binding to the molecular antigen in preference to binding other molecules in the sample, other portions of the molecule or a portion of the vessel or vessels in which the MAD assays are performed. A specific binding interaction discriminates over non-specific binding interactions by about 2-fold or more, often about 10-fold or more, and sometimes about I 00-fold or more.

Each MAD hybrid is selected for specific binding to a molecular antigen and the antigen is selected from a variety of molecules. For example, the molecular antigen sometimes is a portion of, consists of or comprises a nucleic acid (e.g., double-stranded DNA (dsDNA), single-stranded DNA (ssDNA), or RNA), a nucleotide, a nucleotide analog or derivative (e.g., bromodeoxyuridine (BrdU)), an alkyl moiety (e.g., methyl moiety on methylated DNA or methylated histone), an alkanoyl moiety (e.g., an acetyl group of an acetylated protein (e.g., an acetylated histone)), an alkanoic acid or alkanoate moiety (e.g., a fatty acid), a glyceryl moiety (e.g., a lipid), a phosphoryl moiety, a glycosyl moiety or an ubiquitin moiety.

The molecular antigen often consists of or is a portion of a protein, a peptide, or protein. The antigen frequently is a subregion of a protein, such as in the N-terminus, C-terminus, extracellular region, intracellular region, transmembrane region, active site (e.g., nucleotide binding region or a substrate binding region), a domain (e.g., an SH2 or SH3 domain) or a post-translationally modified region (e.g., phosphorylated, glycosylated or ubiquinated region), for example. The antigen sometimes consists of the modification moiety or a portion thereof (e.g., the glycosyl group or a portion thereof) or is the modification moiety in conjunction with amino acids of the protein or peptide to which it is linked (e.g., a phosphoryl group in combination with one or more amino acids of the protein or peptide). In certain embodiments, the protein is a signal transduction factor, cell proliferation factor, apoptosis factor, angiogenesis factor, or cell interaction factor. Examples of cell interaction factors include but are not limited to cadherins (e.g., cadherins E, N, BR, P, R, and M; desmocollins; desmogleins; and protocadherins); connexins; integrins; proteoglycans; immunoglobulins (e.g., ALCAM, NCAM-1 (CD56), CD44, intercellular adhesion molecules (e.g., ICAM-1 and ICAM-2), LFA-1, LFA-2, LFA-3, LECAM-1, VLA-4, ELAM and N-CAM); selectins (e.g., L-selectin (CD62L), E-selectin (CD62e), and P-selectin (CD62P)); agrin; CD34; and a cell surface protein that is cyclically internalized or internalized in response to ligand binding. Examples of signal transduction factors include but are not limited to protein kinases (e.g., mitogen activated protein (MAP) kinase and protein kinases that directly or indirectly phosphorylate it, Janus kinase (JAKI), cyclin dependent kinases, epidermal growth factor (EGF) receptor, platelet-derived growth factor (PDGF) receptor, fibroblast-derived growth factor receptor (FGF), insulin receptor and insulin-like growth factor (IGF) receptor); protein phosphatases (e.g., PTPIB, PP2A and PP2C); GDP/GTP binding proteins (e.g., Ras, Raf, ARF, Ran and Rho); GTPase activating proteins (GAFs); guanine nucleotide exchange factors (GEFs); proteases (e.g., caspase 3, 8 and 9), ubiquitin ligases (e.g., MDM2, an E3 ubiquitin ligase), acetylation and methylation proteins (e.g., p300/CBP, a histone acetyl transferase) and tumor suppressors (e.g., p53, which is activated by factors such as oxygen tension, oncogene signaling, DNA damage and metabolite depletion). The protein sometimes is a nucleic acid-associated protein (e.g., histone, transcription factor, activator, repressor, co-regulator, polymerase or origin recognition (ORC) protein), which directly binds to a nucleic acid or binds to another protein bound to a nucleic acid.

In certain embodiments, the first molecule and the second molecule are in a complex, and sometimes are a non-hybrid nucleic acid and a protein. The first molecule sometimes is a non-hybrid DNA and the second molecule sometimes is a protein; the first molecule sometimes is a protein and the second molecule sometimes is a protein; and sometimes the first molecule and the second molecule are in association with a non-hybrid nucleic acid. In some embodiments, the first binding partner is a protein, the second binding partner is a protein, the first binding partner and the second binding partner is a protein; the first binding partner is an antibody or antibody fragment; the second binding partner is an antibody or antibody fragment; or the first binding partner and the second binding partner is an antibody or antibody fragment.

In specific embodiments, the hybrids generate a detectable signal when two or more hybrids bind to antigens in proximity to one another. For example, the molecules often are between 25 nm to 1,000 nm from one another, sometimes are between 50 nm to 500 nm from one another, and at times are within about 25 nm, about 50 nm, about 75 nm, about 100 nm, about 150 nm, about 200 nm, about 250 nm, about 300 nm, about 350 nm, about 400 nm, about 450 nm, or about 500 nm from one another. The molecules that bind to the hybrids often exist in a complex that comprises other molecules. In the complex, the molecules that bind to the hybrids may be in direct contact with one another or may be indirectly in association with one another as they may be in contact with other molecules of the complex. The term “in association” refers to molecules that directly contact one another or indirectly contact one another by binding to a common molecule or an intermediary group of molecules. A complex can include any of the molecules described above. Two or more hybrids may bind to any combination of molecules in a sample, where the hybrids may bind, for example, a protein, a nucleic acid or one hybrid may bind a protein and another hybrid may bind a nucleic acid. In certain embodiments, one hybrid may bind a protein and another hybrid may bind another protein, where the two proteins sometimes are in association with a nucleic acid that does not bind to a hybrid and is not part of a hybrid (i.e., a non-hybrid nucleic acid).

Each hybrid comprises a binding partner that specifically binds to a molecular antigen described above. The hybrid binding partner and molecule to which it binds can be any binding pair useful for conducting MAD applications, including, but not limited to, antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, nucleic acid/complementary nucleic acid (e.g., DNA, RNA, PNA), and chemical reactive group/complementary chemical reactive group (e.g., sulfhydryl/maleimide, sulfhydryl/haloacetyl derivative, amine/isotriocyanate, amine/succinimidyl ester, and amine/sulfonyl halides). In certain embodiments the binding partner is a cellular binding partner of the molecule containing the antigen. For example, where the molecular antigen is a subregion of a receptor protein kinase such as EGF receptor, the binding partner is EGF or a functional fragment thereof; where the molecular antigen is a nucleic acid, the binding partner sometimes is a transcription factor or histone or a functional portion thereof; or where the molecular antigen is a glycosyl moiety, the binding partner sometimes is a glycosyl binding protein or a portion thereof.

The binding partner often is an antibody or a fragment thereof. Antibodies sometimes are IgG, IgM, IgA, IgE, or an isotype thereof (e.g., IgG₁, IgG₂ₐ, IgG_{2b} or IgG₃), sometimes are polyclonal or monoclonal, and sometimes are chimeric, humanized or bispecific versions of such antibodies. Polyclonal and monoclonal antibodies that bind specific antigens are commercially available, and methods for generating such antibodies are known. In general, polyclonal antibodies are produced by injecting an isolated antigen into a suitable animal (e.g., a goat or rabbit); collecting blood and/or other tissues from the animal containing antibodies specific for the antigen and purifying the antibody. Methods for generating monoclonal antibodies, in general, include injecting an animal with an isolated antigen (e.g., often a mouse or a rat); isolating splenocytes from the animal; fusing the splenocytes with myeloma cells to form hybridomas; isolating the hybridomas and selecting hybridomas that produce monoclonal antibodies which specifically bind the antigen (e.g., Kohler & Milstein, Nature 256:495-497 (1975) and StGroth & Scheidegger, J. Immunol Methods 5:1-21 (1980)). Examples of monoclonal antibodies are anti-MDM-2 antibodies, anti-p53 antibodies (pAB421, DO-1, and an antibody that binds phosphoryl-ser15), anti-dsDNA antibodies and anti-BrdU antibodies, are described hereafter.

Methods for generating chimeric and humanized antibodies also are known (see, e.g., U.S. Pat. No.5,530,101 (Queen, et al.), U.S. Pat. No.5,707,622 (Fung, et al.) and U.S. Pat. Nos. 5,994,524 and 6,245,894 (Matsushima, et al.)), which generally involve transplanting an antibody variable region from one species (e.g., mouse) into an antibody constant domain of another species (e.g., human). Antigen-binding regions of antibodies (e.g., Fab regions) include a light chain and a heavy chain, and the variable region is composed of regions from the light chain and the heavy chain. Given that the variable region of an antibody is formed from six complementarity-determining regions (CDRs) in the heavy and light chain variable regions, one or more CDRs from one antibody can be substituted (i.e., grafted) with a CDR of another antibody to generate chimeric antibodies. Also, humanized antibodies are generated by introducing amino acid substitutions that render the resulting antibody less immunogenic when administered to humans.

The binding partner sometimes is an antibody fragment, such as a Fab, Fab′, F(ab)′₂, Dab, Fv or single-chain Fv (ScFv) fragment, and methods for generating antibody fragments are known (see, e.g., U.S. Pat. Nos. 6,099,842 and 5,990,296 and PCT/GB00/04317). In some embodiments, a binding partner in one or more hybrids is a single-chain antibody fragment, which sometimes are constructed by joining a heavy chain variable region with a light chain variable region by a polypeptide linker (e.g., the linker is attached at the C-terminus or N-terminus of each chain) by recombinant molecular biology processes. Such fragments often exhibit specificities and affinities for an antigen similar to the original monoclonal antibodies. Bifunctional antibodies sometimes are constructed by engineering two different binding specificities into a single antibody chain and sometimes are constructed by joining two Fab′ regions together, where each Fab′ region is from a different antibody (e.g., U.S. Pat. No. 6,342,221). Antibody fragments often comprise engineered regions such as CDR-grafted or humanized fragments. In certain embodiments the binding partner is an intact immunoglobulin, and in other embodiments the binding partner is a Fab monomer or a Fab dimer.

Each hybrid comprises a nucleic acid in addition to a binding partner. The nucleic acid often is a deoxyribonucleic acid (DNA) and sometimes is a ribonucleic acid (RNA) or a derivative or analog thereof (e.g., a peptide nucleic acid (PNA)). Standard methods are utilized for generating synthetic nucleic acids, where DNA sometimes is synthesized using an ABI™3900 High Throughput DNA Synthesizer, which is available from Applied Biosystems (Foster City, Calif.), and PNA and other nucleic acid analogs or derivatives are generated as exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; 5,614,622; 5,739,314; 5,955,599; 5,962,674; 6,117,992; WIPO publications WO 00/56746, WO 00/75372 and WO 01/14398, and related publications. In certain embodiments, one 3′ nucleotide or two, three, four or five 3′ nucleotides are nucleotide analogs useful for inhibiting exonuclease digestion of the hybrid nucleic acids in certain samples. In other embodiments, some nucleotides are fluorescent analogs of naturally occurring nucleotides. Examples of fluorescent nucleotide analogs are 2-amino purine (e.g., 2-amino-adenosine), pyrrolo-C, 6-MAP, and furano-dT (for other examples, see http address www.glenresearch.com/GlenReports/BR1 5-13.html).

The nucleic acid in each hybrid is the means by which the presence of each molecular antigen to which a binding partner specifically binds is identified. The nucleic acid often is single-stranded, where the single-stranded nucleic acid often hybridizes to single-stranded nucleic acid linked to another hybrid and the resulting hybridization product or an extension product generated from the hybridization product is detected. In some embodiments, a single-stranded nucleic acid is detected directly (e.g., by PCR). In some embodiments, the hybrid nucleic acid is double-stranded and is detected by any suitable detection technique, such as by PCR or a nucleic acid binding agent linked to a detectable label.

The hybrid nucleic acid is designed with an appropriate number of nucleotides for detection, and methods described hereafter are routinely performed to optimize the number of nucleotides in each hybrid nucleic acid. Each hybrid nucleic acid often is about 2 to about 100 nucleotides, about 10 to about 90 nucleotides, or about 30 to about 80 nucleotides, and sometimes is about 45 to about 70 nucleotides, about 50 to about 70 nucleotides, or about 65 to about 70 nucleotides in length.

In embodiments where a single-stranded hybrid nucleic acid is capable of forming a hybridization product with a single-stranded nucleic acid of another hybrid, the nucleic acid often is about 55 nucleotides in length, and sometimes is about 40 nucleotides, about 45 nucleotides, about 50 nucleotides, 51, 52, 53, 54, 55, 56, 57, 58, or 59 nucleotides, about 60 nucleotides, about 65 nucleotides, or about 70 nucleotides in length. Hybrid nucleic acids in the resulting hybridization product sometimes are entirely overlapping (e.g., every nucleotide in one nucleic acid is complementary to a nucleotide in the other nucleic acid), and often, hybrid nucleic acids are partially overlapping (e.g., the hybridization product comprises an overlapping region where nucleotides are complementary and a non-overlapping region where nucleotides are not complementary). In the latter embodiments, the hybridization product is partially single-stranded and partially double-stranded, and the overlapping region and non-overlapping region are of a length that facilitates specific detection and maintains a detectable portion of the hybridization product after MAD assay steps are completed. Methods described hereafter are useful for optimizing the length of the overlapping region of hybrid nucleic acids. Overlapping regions frequently are between about 3 to about 20 nucleotides, sometimes about 5 to about 15 nucleotides, sometimes about 3 to about 6 nucleotides, about 4 to about 5 nucleotides and sometimes 5 nucleotides in length. The overlapping region is located in an appropriate portion of the hybrid nucleic acid that allows for detectable hybridization, often at the terminus or near the terminus opposite the end joined to the binding partner. The nucleotide sequences in the hybrid nucleic acids that hybridize to one another often are perfectly complementary, and sometimes are not perfectly complementary but allow for sufficient hybridization to form a stable hybridization product that can be extended and/or detected. The non-overlapping region often is about 30 to about 50 nucleotides, at times is about 40 to about 45 nucleotides, and sometimes is 38, 39, 40, 41, 42, 43, 44, 45, 46, or 47 nucleotides in length. The non-overlapping region often includes subsequences having specific functions. In an embodiment, a subsequence in the non-overlapping region sometimes contributes to a binding determinant for a nucleic acid binding agent, where the binding determinant is formed partially by the overlapping region and partially by the non-overlapping region. In some embodiments, a unique nucleotide subsequence is incorporated into hybrid nucleic acids for detection of hybridization products or extension products in multiplexed assays described hereafter. In the latter embodiments, the hybrid nucleic acid often includes a subsequence that hybridizes to another hybrid nucleic acid and another subsequence that is unique to that hybrid nucleic acid and can be detected in a multiplex assay (e.g., by an amplification process or by a nucleic acid binding agent). In other embodiments, the length of the entire nucleic acid sometimes is designed as a molecular ruler for estimating the distances between proximal molecules detected by MAD assays, as described hereafter.

Hybrid nucleic acids are linked to the binding partner using standard methods for generating covalent or non-covalent linkages (e.g., Hendrickson, et al., Nucleic Acids Res. (1995) 23:522-529; Joerger, et al., Clinical Chemistry (1995) 41:1371-1377; Wu, et al., Lttrs. App. Microbiology (2001) 32:321-325 and U.S. Pat. Nos. 5,635,602 (Cantor, et al.); 5,665,539 (Sano, et al.); 4,340,535 (Voisin, etal.); and 6,004,554; 5,855,866; 5,965,132; 5,776,427 ; 5,863,538; 5,660,827 and 6,051,230 (Thorpe, et al.). A nucleic acid often is linked by covalent attachment to the binding partner by chemically modifying the nucleic acid and the binding partner molecule and then joining them to one another. The nucleic acid often is derivitized at the 5′ end with a sulfhydryl- or amine-reactive chemical moiety, including, but not limited to a maleimide or haloacetyl sulfhydryl-reactive moiety or a isotriocyanate, succinyl ester or sulfonyl halide amine-reactive moiety. Where the nucleic acid is derivitized with a sulfhydryl-reactive moiety, the binding partner, often an antibody, that includes one or more sulfhydryl moieties often is contacted with a reducing agent (e.g., dithiothreitol (DTT)) for coupling the nucleic acid. In an embodiment, the binding partner is an antibody and is derivitized with maleimide using sulfo-SMCC, and an aminoallyl-modified nucleic acid is converted to an acetylthioacetyl modified nucleic acid using a SATA reagent. Conditions for the coupling reaction often are selected to control the stoichiometric ratio of nucleic acid to binding partner in each hybrid product. The derivitized nucleic acid and antibody sometimes are ligated in a stoichiometric ratio of about one to one to form the hybrid, although other stoichiometric ratios sometimes are utilized, such as between about 0.8-1.2 to one antibody to nucleic acid or 0.8-1.2 nucleic acid to antibody. The reaction product often is purified by an appropriate separation technique, such as gel filtration high performance liquid chromatography (HPLC), and a suitable technique for detecting fractions containing the hybrid often is utilized, such as spectrophotometric scans at absorbance ratios of 260 nm/280 nm. Further purification that removes unreacted and free nucleic acids from the hybrid sometimes is performed by utilizing micro-concentrators (e.g., Microcon 100) or other appropriate separation devices. Methods for linking binding partners by non-covalent linkage to nucleic acids sometimes is accomplished by biotinylating the binding partner and the nucleic acid and linking them via avidin (e.g., Sano, et al., Proc. Natl. Acad. Sci. USA (1995) 92:272-275), and where the binding partner specifically is an antibody, by biotinylating the nucleic acid and linking it to the antibody via a protein A/streptavidin chimera (Sano, et al., Science (1992) 258:120-122).

A hybrid sometimes comprises a molecule other than the detection nucleic acid and the binding partner. In one embodiment, the hybrid includes a spacer molecule located between the binding partner and the hybrid nucleic acid. Methods for joining spacer molecules to nucleic acids and binding partner molecules are known in the art, and an example of a spacer molecule is a polyethylene glycol (PEG) molecule. In certain embodiments, a hybrid is in association with a solid support, which is described in further detail hereafter. In some embodiments, a hybrid comprises a detectable label linked to the nucleic acid and/or the binding partner. Examples of detectable labels are fluorescent labels such as fluorescein, rhodamine, and others (see, e.g., Anantha, etal., Biochemistry (1998) 37:2709-2714; and Qu & Chaires, Methods Enzymol. (2000) 321:353-369); a radioactive isotope (e.g., 1-1 25,1-131, S-35, P-31, P-32, C-14, H-3, Be-7, Mg-28, Co-57, Zn-65, Cu-67, Ge-68, Sr-82, Rb-83, Tc-95m, Tc-96, Pd-103, Cd-109, and Xe-127); a light scattering label (see, e.g., U.S. Patent No. 6,214,560, and commercially available from Genicon Sciences Corporation, San Diego, Calif.); an enzymic or protein label (e.g., green fluorescence protein (GFP) or peroxidase); or another chromogenic label or dye (e.g., cyanine).

MAD Assays

MAD assays are performed by contacting a sample with two or more hybrids and detecting hybrids bound to molecular antigens of interest. The sample sometimes is a cell, a group of cells, sometimes is prepared from a cell or group of cells, sometimes is a purified fraction from a cell preparation, and sometimes is a purified molecule, for example. Any appropriate cell may be utilized for MAD assays, including mammalian (e.g., human), insect, yeast, fungal, or bacterial cells. Cells from multicellular organism (e.g., insects and mammals) sometimes are derived from specific portions of the organism (e.g., specific tissues, organs, or fluids). Cells are contacted by hybrids in vitro or in vivo, and are contacted by hybrids when in suspension or when attached to a solid surface. Cells sometimes are not significantly modified during the process, and sometimes are fixed to a solid support and made permeable using standard methods (see, e.g., Melan,

Methods Mol. Biol. 34:55-66 (1994)). In some embodiments, samples are prepared by lysing cells to form cell lysates. In embodiments involving permeable or lysed cells, hybrids often are contacted with hybrids after cells are permeable or lysed, and sometimes cells are contacted with hybrids before or during permeablization or lysis. In other embodiments, components of cells are separated from whole cells, permeable cells, or cell lysates before a sample is contacted with hybrids. For example, chromatin DNA cross-linked to proteins sometimes is separated from cell lysates before the sample is contacted with hybrids in MAD chromatin immunoprecipitation (MAD-ChIP) assays described hereafter.

MAD assays are conducted in any suitable or convenient environment or system for conducting assays. Examples of systems include but are not limited to microtiter plates (e.g., 96-well or 384-well plates), test tubes, silicon chips having molecules immobilized thereon and optionally oriented in an array (see, e.g., U.S. Pat. No. 6,261,776 and Fodor, Nature 364: 555-556 (1993)), and microfluidic devices (see, e.g., U.S. Pat. Nos. 6,440,722; 6,429,025; 6,379,974; and 6,316,781). The system can include attendant equipment for carrying out the assays, such as signal detectors, robotic platforms, and pipette dispensers. The sample and hybrids are contacted under conditions suitable for allowing signal detection when hybrids bind to molecular antigens in the sample. For example, in embodiments where a signal is generated from a hybridization product between two hybrids having complementary single-stranded nucleic acids, appropriate conditions allow formation of the hybridization product when the two hybrids are bound to their respective molecular antigens. Cell samples or samples derived from cells often require no modification before they are contacted with a hybrid, although sample preparation procedures sometimes are performed before, during, or after the sample is contacted by the hybrids. In certain embodiments, a cell lysate is mixed with a buffer solution to stabilize the pH of the sample and/or the sample is contacted with one or more protease inhibitor molecules that slow or halt the breakdown of certain proteins in the sample. Assay components are contacted and mixed with one another in any suitable or convenient manner, such as by oscillating a vessel, subjecting a vessel to a vortex generating apparatus, repeated mixing with a pipette or pipettes, or by passing fluid containing one assay component over a surface having another assay component immobilized thereon, for example. Samples, hybrids and other assay components (e.g., a signal enhancing nucleic acid and/or a nucleic acid binding agent) are contacted with one another in any order that allows for specific and selective detection of the molecule or molecules by the hybrids. For example, MAD assays sometimes are conducted by adding one hybrid to the system before another hybrid is added, and sometimes hybrids are added at one time. In some embodiments, sample is added before a hybrid and sometimes a hybrid is added before a sample. Other components, such as a signal enhancing nucleic acid or a nucleic acid binding agent are added to the system before, at the same time, or after a hybrid or sample is added to the system.

In some embodiments, the hybridization product is extended by contacting it with nucleotides and a polymerase, such as a Klenow fragment or Sequenase™ polymerase. In some embodiments, the hybridization product is detected without extending it.

One or more other nucleic acids that enhance detection of a hybridization product in MAD assays sometimes are utilized, and sometimes are referred to herein as “signal enhancer nucleic acids.” Signal enhancer nucleic acids are applicable to many of the embodiments described herein. A signal enhancer nucleic acid specifically hybridizes to a single stranded portion of the first nucleic acid or second nucleic acid in the hybridization product, where the 5′ end of signal enhancer nucleic acid abuts the 3′ end of the other of the first nucleic acid or the second nucleic acid. As with hybrid nucleic acids, the nucleotide sequence of a signal enhancer nucleic acid often is perfectly complementary to the nucleotide sequence in the hybrid nucleic acid, but in some embodiments, it need not be entirely complementary so long as a hybridization product or extension product can be detected. A signal enhancer nucleic acid sometimes is about 15 to about 40 nucleotides in length, sometimes extends to the 5′ end of the first nucleic acid or second nucleic acid, and often does not extend to the 5′ end of the first nucleic acid or second nucleic acid. The term “abuts” often refers to a configuration in which there is no gap between the 5′ end of the signal enhancer nucleic acid and the 3′ end of the first or second nucleic acid, and sometimes refers to a configuration in which there is a gap of one or two nucleotides between the 5′ end of the signal enhancer nucleic acid and the 3′ end of the first or second nucleic acid. Without being bound by theory, it is expected that the signal enhancer nucleic acid facilitates binding of a polymerase used to extend the hybridization product. A signal enhancer nucleic acid is added at any stage of a MAD application, including, but not limited to, prior to the time the sample is contacted with one or more hybrids (e.g., a signal enhancer nucleic acid sometimes is contacted with a hybrid to which it hybridizes before the hybrid and signal enhancer nucleic acid are contacted with the sample), at the same time the sample is contacted with hybrids, after molecules and hybrids are diluted or washed and before a hybridization product is extended, for example.

In some embodiments, two or more signal enhancer nucleic acids are added. For example, a first signal enhancer nucleic acid specifically hybridizes to a single stranded region of the first nucleic acid of the first hybrid and the second signal enhancer nucleic acid specifically hybridizes to a single stranded region of the second nucleic acid of the first hybrids when the first nucleic acid and the second nucleic acid are in a hybridization product. The 5′ end of the first signal enhancer nucleic acid abuts the 3′ end of the second nucleic acid in the second hybrid and the 5′ end of the second signal enhancer nucleic acid abuts the 3′ end of the first nucleic acid in the first hybrid. In embodiments where two or more signal enhancer nucleic acids are utilized, the hybridization product is extended by a polymerase capable of extending the complex (e.g., strand displacement polymerase or a nick translation polymerase). In some embodiments, two or more signal enhancer nucleic acids are utilized and they extend to or within a few nucleotides of the 5′ end of the first nucleic acid and second nucleic acid (e.g., within 1, 2, 3, 4 or 5 nucleotides of the 5′ end). In such embodiments the hybridization product sometimes is not extended and the complex formed by the first hybrid nucleic acid, second hybrid nucleic acid, first enhancer nucleic acid and second enhancer nucleic acid is detected directly by a nucleic acid binding agent. In certain embodiments, a ligase is added to ligate a signal enhancer nucleic acid to the first and/or second hybrid nucleic acid, and the resulting ligation product sometimes is detected by a nucleic acid binding agent or by an amplification process such as PCR.

MAD processes often include one or more steps that lower signals attributed to non-specific interactions, also referred to as “background signals.” The sample and the hybrids sometimes are diluted in assay embodiments after the hybrids and sample are contacted, where the molecules and hybrids at this stage of the assay are not in association with a solid phase. In some embodiments, one or more assay components are immobilized to a solid phase after dilution (e.g., a system comprising a hybrid linked to magnetic beads is diluted after the second hybrid and the sample is added, and then the magnetic beads are attracted to a magnetic solid phase). Dilution often is 1:5 or more, and sometimes is 1:10 or more, 1:50 or more, 1:100 or more, 1:500 or more, 1:1000 or more, 1:5000 or more, or 1:10000 or more. Dilution often is performed by adding a volume of the same or similar buffer solution used for the assay. An agent that inhibits non-specific interactions between molecules in the sample (e.g., a blocking agent) sometimes is included at the time of dilution, or before or after dilution in certain embodiments. Blocking agents are known, and included, but are not limited to, bovine serum albumin (BSA), DNA (e.g., sheared salmon sperm DNA), RNA (e.g., yeast RNA), serum from a variety of animals, casein, IgG (e.g., species-specific IgG), avidin, biotin, gelatin, citraconic anhydride and others The sample and hybrids sometimes are washed after a sample is contacted with one or more hybrids in heterogeneous assay embodiments where a molecule and/or hybrid is in association with a solid phase. Washing is performed in any manner suitable for conducting assays, and can be carried out with convenient volumes and in convenient numbers so long as a hybridization products or extension products can be detected when they are present in the assay system. Dilution and/or washing often are performed before the hybridization product is extended and before the hybridization product or extension product is detected. Control assays described hereafter are useful for ruling out false positive signals and subtracting background signals, and often are utilized when the amount or concentration of the antigen is quantified in the sample.

The presence or absence of a MAD hybridization product or extension product is detected in a sample using any convenient procedure. One suitable detection procedure is an amplification procedure such as the polymerase chain reaction (PCR). Another amplification procedure is transcription-mediated amplification (TMA), in which two enzymes are used in an isothermal reaction to produce amplification products detected by light emission (see, e.g., Biochemistry 1996 Jun 25;35(25):8429-38 and http address www.devicelink.com/ivdt/archive/00/1 1/007.html). Standard PCR processes are known (e.g., U.S. Pat. Nos. 4,683,202; 4,683,195; 4,965,188; and 5,656,493). Generally, PCR processes are performed in cycles, where each cycle includes heat denaturation, in which hybrid nucleic acids dissociate; cooling, in which primer oligonucleotides hybridize; and extension of the oligonucleotides by a polymerase (i.e., Taq polymerase). An example of a PCR cyclical process is treating the sample at 95° C. for 5 minutes; repeating forty-five cycles of 95° C. for 1 minute, 59° C. for 1 minute, 10 seconds, and 72° C. for 1 minute 30 seconds; and then treating the sample at 72° C. for 5 minutes. Multiple cycles frequently are performed using a commercially available thermal cycler. PCR amplification products sometimes are stored for a time at a lower temperature (e.g., at 4° C.) and sometimes are frozen (e.g., at −20° C.) before analysis.

PCR amplification products are detected by any suitable manner. For example, PCR application products in a sample sometimes are resolved and detected by gel electrophoresis (e.g., plate or capillary gels composed of polyacrylamide or agarose), where bands corresponding to amplification products are resolved by size and visualized by a light-emitting dye that intercalates with nucleic acid products in the gel (e.g., ethidium bromide). In one embodiment, PCR amplification products are quantified by determining signal intensities of bands on a gel (e.g., by scanning the gel with a commercially available densitometer). In some embodiments, amplification products are quantified by hybridization techniques (e.g., performing real time (RT)-PCR using commercially available TaqMan® (Applied Biosystems, Inc.) or LUX® (Invitrogen, Inc.) products). In the latter embodiment, a double-labeled oligonucleotide complementary to a PCR product often is utilized in the quantification procedure, where one or both labels often is a fluorescent molecule (e.g., a carboxyfluorescein dye (FAM™ or FAM-X™) at the 5′ end of the oligonucleotide and a carboxytetramethylrhodamine dye (TAMRA™) at the 3′ end of the oligonucleotide (see e.g., these and other fluorescent dyes are commercially available at http address www.synthegen.com). Lower limits of the PCR detection process sometimes are determined by serially diluting a sample and determining the lowest detectable antigen concentration, number of antigen molecules, or cells in a sample. (e.g., lower limits of 10⁶, 10⁵, 10⁴, 10³, 10² and 10¹ molecules may be detected).

The hybridization product sometimes is detected directly by an amplification procedure such as PCR (e.g., using oligonucleotides complementary to overlapping regions in the hybridization product) or by a nucleic acid binding protein linked to a detectable label (e.g., the nucleic acid binding protein binds to a nucleotide sequence in the overlapping region). In other embodiments, the hybridization product is extended by a polymerase and the extension product is detected, often by PCR or by a nucleic acid binding protein linked to a detectable label. In embodiments where the hybridization product is extended, naturally occurring nucleotides frequently are utilized for the extension reaction (e.g., thymidine, adenine, cytosine, and guanine), and sometimes one or more nucleotide analogs or modified nucleotides are utilized (e.g., a nucleotide derivitized with biotin, avidin, streptavidin or a detectable label). For embodiments in which an extension product is generated, the extension reaction sometimes is incorporated within the PCR process by the same enzymes utilized for PCR, and often the extension reaction is a process separate from PCR. For example, the extension step sometimes is performed at an ambient temperature (e.g., at about 37° C. using an ambient temperature polymerase such as Klenow fragment) or at a higher temperature at which PCR is performed (e.g., at about 55° C. to 80° C. (e.g., sometimes about 72° C.) using a thermal stable polymerase such as T aquaticus (Taq) polymerase). The extension reaction also can be performed using either enzyme at lower temperatures, such as 4° C. The extension product sometimes is immobilized to a solid support before, during, or after detection, as described hereafter.

Another suitable detection procedure involves contacting the sample with a nucleic acid binding agent that specifically binds to a nucleotide sequence in a hybrid nucleic acid, hybridization product nucleic acid or extension product nucleic acid. The nucleic acid binding agent sometimes is referred to hereafter as a “reporter molecule,” as it often comprises a detectable label described herein. Many appropriate molecules that specifically bind to a hybrid nucleic acid are available, and include for example, a complementary nucleic acid linked to a detectable label, a nucleic acid for detecting and recovering extended overlaps by triplex formation (see, e.g., Schleup & Cooney, Nucl. Acids Res. 26:4524-4528 (1998)), or a nucleic acid binding protein linked to a detectable label. Examples of nucleic acid binding proteins are a transcription factor, a repressor protein (e.g., a lac operon repressor (Belmont, et al., Methods Cell Biol. 58:203-222 (1999))), an activator, a co-regulator, a histone, a gal4 protein, a tus protein, or a nucleic acid binding fragment of the foregoing. Nucleotide sequences to which the nucleic acid binding proteins bind are known and are readily adapted to the design of hybrid nucleic acids, and sequence variations and shorter sequences can be optimized using optimization processes described herein. Often, a nucleic acid binding protein is heterologous with respect to the molecule or molecules being targeted (e.g., the molecules are in a human biological sample and a bacterial lacO sequence formed in the hybridization product or extension product is bound by a bacterial lac repressor protein or functional fragment thereof). A nucleic acid binding agent often binds to a double-stranded nucleic acid, and sometimes binds to a single-stranded or partially double stranded and single stranded nucleic acid. The nucleic acid binding agent often binds to an extension product generated from a hybridization product, and in some embodiments, binds to an unextended hybridization product (e.g., frequently binding to an overlapping, double stranded region of the hybridization product).

The detectable label sometimes is linked to the nucleic acid binding agent by a covalent attachment or a non-covalent attachment. The label sometimes is linked to the nucleic acid binding agent before, during, or after the hybridization product or extension product is contacted with the nucleic acid binding agent. Examples of detectable labels are described above and methods for joining them to reporter nucleic acids or nucleic acid binding proteins are known. Detectable labels often are joined to nucleic acid binding agents by derivitizing the label with one linking molecule (e.g., avidin or streptavidin) and derivitizing the nucleic acid binding agent with another complementary linking molecule (e.g., biotin), as described in further detail herein. In an embodiment, the molecule that binds to the hybrid nucleic acid is joined to biotin, the label is joined to avidin, and a hybridization product or extension product is contacted with the biotinylated molecule before the sample is contacted with the avidin-conjugated label. The detectable label is detected by using any appropriate method known, and often, the sample is treated to separate reporter molecules bound to MAD assay components from unbound reporter molecules such that reporter molecules bound to MAD assay components are detected. Such separation steps often are performed by washing a sample with a solution that carries unbound reporter molecules away from reporter molecules bound to MAD assay components. For example, the hybridization product or extension product to which a nucleic acid binding agent is bound sometimes is in association with a solid phase and the solid phase is washed to remove unbound nucleic acid binding agent before the detectable label on the nucleic acid binding agent is detected.

In certain embodiments, a fluorescent signal generated by a label on the nucleic acid binding agent bound to the hybridization product or extension product is detected. Fluorescent labels, apparatus and methods of detecting fluorescent signals are known (e.g., fluorescence detector or scattered light detector and methods of using them). Methods for detecting fluorescent signals include, but are not limited to, directly detecting a fluorescent signal, detecting a quenched signal, detecting fluorescence polarization, and detecting a fluorescence energy resonance transfer (FRET) signal, for example. Fluorescent reporter molecules utilized to detect MAD assay components are useful for determining whether molecules are in proximity in a cell or on a cell surface (e.g., in situ detection techniques are described in greater detail hereafter) and are useful for detecting molecular antigens in samples prepared from cells (e.g., cell lysate and chromatin DNA preparations). The reporter molecules also are useful for flow cytometry procedures, such as flow microfluorimetry (FMF) and fluorescence activated cell sorting (FACS); U.S. Pat. Nos. 6,090,919 (Cormack, etal.); 6,461,813 (Lorens); and 6,455,263 (Payan)). In certain embodiments, cells or samples are contacted with a reporter molecule that includes a lac repressor protein or fragment thereof linked to biotin and then are contacted by avidin-linked fluorescein isothiocyanate (FITC). In other embodiments, cells or samples are contacted with a reporter molecule comprising a lac repressor protein or fragment thereof linked to an enzyme such as horse radish peroxidase (HRP). The latter embodiments are useful for detecting proximal antigens on a membrane (e.g., in a Western assay, where a membrane is contacted with the reporter molecule), in an enzyme linked immunosorbant (ELISA) assay, and in plate reader/robotic methods.

Yet another suitable detection procedure is extending a hybridization product having a non-overlapping region with nucleotides joined to a detectable label or a linking moiety such as biotin, avidin, or streptavidin. Such extension products often are immobilized to a solid support as described in greater detail hereafter, and the immobilized molecules can be detected on the solid support or detected after they are eluted from the solid support, for example.

MAD assays sometimes are performed in conjunction with one or more control assays, in which one or more of the molecular antigens is not present, one or more molecular antigens is modified such that binding to a hybrid is impaired or nullified, or one or more of the hybrids is not contacted with the sample. In other controls, the sample is not exposed to an exogenous molecule or a specific condition. Such controls are useful for determining whether any signal (e.g., PCR amplification products or from a detectable label linked to a nucleic acid binding molecule) corresponds to nonspecific binding of a hybrid to a reaction vessel or nonspecific binding to components in the sample other than the molecular antigens to which the hybrid specifically binds. The controls also are useful for determining background signal levels for signal quantification and for identifying any false positive results. Another control experiment involves contacting hybrids with known amounts or concentrations of a molecular antigen to which a hybrid binds, which is useful for quantifying the amount of a particular antigen or the amount of a complex comprising the antigen in a sample. Non-specific binding also is mitigated in certain embodiments by heating a sample after hybridization products form. In such embodiments, samples are briefly heated to 50° C., for example, and then hybridization products or extension products are detected shortly thereafter.

A procedure sometimes is performed to determine the limit of detection for a MAD assay, which often is expressed in terms of an antigen concentration, antigen amount, or number of cells. The sample, which has a known antigen amount, antigen concentration, or number of cells, is serially diluted and the lowest antigen amount, antigen concentration, or number of cells for which a detectable signal is detected is determined as the lower limit of detection. MAD assays may detect at lower limits about 10⁶, about 10⁵, about 10⁴, about 10³, about 10² or about 10¹ molecules per sample.

MAD assays sometimes are utilized to optimize MAD hybrids and assay conditions. For example, in an embodiment where a sample is contacted by hybrids capable of forming a hybridization product, hybrids having different nucleic acid sequences, different lengths or different lengths of overlapping regions or non-overlapping regions, for example, sometimes are screened to determine which hybrids are optimal for assaying the molecular antigens to which they bind. In this procedure, a sample is divided and contacted with different hybrids (e.g., the length of the overlapping region is of varying lengths or a different binding partner binds to a different antigen in a molecule), signals corresponding to hybridization products are detected, and the signals generated for the divided samples are compared to determine which of the hybrids are optimal for antigen detection. This procedure and similar procedures are useful for optimizing the overall length and sequence of hybrid nucleic acids, length and sequence of an overlapping region and/or non-overlapping region, type and size of a binding partner protein, type of a binding partner antibody or type and size of an antibody fragment, type and size of nucleic acid binding protein or fragment thereof, type of detectable label and type of linkage of the label to a nucleic acid binding protein, concentration of reagents utilized (e.g., concentration), dilution conditions, blocking agent concentrations, wash conditions, and other assay conditions (e.g., amount and type of buffer or protease inhibitors). In specific embodiments, hybrid nucleic acids having different sequences and lengths of overlapping and non-overlapping regions are hybridized to one another at low concentrations and higher concentrations to mimic hybrids in solution alone and hybrids bind to proximal antigens, respectively, to optimize hybrid nucleic acid length and composition.

In certain MAD assay embodiments, the signal generated by a hybrid nucleic acid, a hybridization product or extension product sometimes is quantified (e.g., the hybridization product per se or an extension product derived from the hybridization product). In embodiments where the hybridization product, extension product or hybrid nucleic acid is detected by PCR, PCR amplification products are quantified using standard techniques (e.g., RT-PCR processes). In other embodiments, a signal generated by a detectable label linked to a nucleic acid binding protein that binds to the hybrid nucleic acid, hybridization product or extension product is quantified (e.g., a fluorescent label often is detected utilizing a detector and a photomultiplier, both of which are commercially available). Control assays often are performed with known antigen amounts or concentrations, no antigen or no detection agent(s) to quantify antigen according to the signal generated by PCR or the detectable label in the sample.

The distance between two molecular antigens is determined in certain MAD assay embodiments. In such embodiments, a sample is contacted with hybrids having nucleic acids capable of forming a hybridization product when the molecular antigens to which they bind are in proximity in the sample, and where a hybridization product is detected, the distance between the molecular antigens is determined by an appropriate calculation. The length of the hybridization product is calculated in a variety of manners, sometimes by summing the number of nucleotides in the first hybrid nucleic acid and the second hybrid nucleic acid, subtracting half of the nucleotides participating in an overlapping region of the hybridization product, and multiplying the resulting number of nucleotides by the approximate 3.4 Å length of each nucleotide. For example, where the first hybrid and second hybrid nucleic acids are 55 nucleotides in length and the overlapping region is 15 base pairs in length, the hybrid length is 95 nucleotides, and the 95 nucleotides multiplied by 3.4 Å yields a hybridization product 323 Å in length. The length of a linker moiety between the binding partner and the nucleic acid of each hybrid also sometimes is added, especially if the linker is longer than 5 Å. Diameters for binding partners often are known and can be calculated using known techniques if not known (e.g., sedimentation processes or gel filtration processes using standard molecules with known diameters). For example, a Fab′ antibody fragment binding partner is approximately 50 Å in diameter, and therefore the distance between two antigens bound to two Fab′ fragments linked by a 323 Å hybridization product is approximately 423 Å. This distance value is subject to some fluctuation and is dependent, for example, upon the orientation of the Fab′ fragments, which have a diameter of approximately 50 Å if they are displayed outward relative to the hybridization product but have a diameter of approximately 40 Å if they displayed inward. Thus, the distance calculation can vary by about 20 Å in certain circumstances, and possibly more or less in other circumstances.

Secondary MAD Reagents and Assays

In certain embodiments, MAD assay hybrids are utilized as secondary detection agents. In such embodiments, one or more primary binding partners (e.g., an antibody or antibody fragment), which specifically binds to a binding region in a molecule and is not linked to a nucleic acid, is contacted with a sample before, concurrently, or after the sample is contacted with one or more MAD assay hybrids. In these embodiments, each MAD assay hybrid includes a secondary binding partner (e.g., an antibody or antibody fragment) that specifically binds to a region of the primary binding partner and thus acts as a secondary detection agent. In certain embodiments, two or more primary binding partners specifically bind to different molecular antigens in the same molecule or different molecules (e.g., useful for MAD-ChIP assays) or a primary binding partner specifically binds to one molecular antigen (e.g., useful for determining multimerization of a molecule). The primary binding partner and hybrid binding partner are selected from any useful binding pair, including, but not limited to, antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, nucleic acid/complementary nucleic acid (e.g., DNA, RNA, PNA), and chemical reactive group/complementary chemical reactive group (e.g., sulfhydrylmaleimide, sulfhydryl/haloacetyl derivative, amine/isotriocyanate, amine/succinimidyl ester, and amine/sulfonyl halides). In certain embodiments, the primary binding partner is an antibody and the MAD assay hybrid specifically binds to an Fc region of a primary antibody. In the latter embodiment, such a MAD assay hybrid can provide an advantage of universally binding any primary antibody from a particular species (e.g., one MAD assay hybrid can be provided that specifically binds to any goat primary antibody and another MAD assay hybrid can be provided that binds to any mouse primary antibody).

In specific embodiments, the two or more primary binding partners are antibodies derived from a different animal species (e.g., hamster, rabbit, mouse, rat, chicken or goat), and are derived directly (e.g., a goat antibody is isolated from a goat) or indirectly (e.g., a goat antibody is produced by recombinant techniques in bacteria). In other embodiments, each primary antibody is a monoclonal antibody having a unique isotype (e.g., IgG₁ or IgG₂) and the secondary MAD hybrid is an antibody that specifically binds to the Fc region of the specific isotype primary antibody. Many of the embodiments disclosed herein for using MAD hybrids as primary detection agents are applicable to using MAD hybrids as secondary detection agents. Embodiments in which MAD assay hybrids are utilized as secondary detection agents are applicable to any of the assay embodiments discussed herein. For example, MAD assay hybrids that directly bind to molecular antigens are replaced with a different set of agents comprising one or more primary binding agents that specifically bind to molecular antigens (e.g., monoclonal antibodies of different isotypes) and one or more secondary MAD assay hybrids that specifically bind to the one or more primary binding agents (e.g., each hybrid comprises an isotype specific antibody linked to a nucleic acid).

Thus, provided herein is a method for detecting a first molecule and a second molecule in a sample, which comprises contacting the sample with a first primary binding agent and a second primary binding agent, wherein the first primary binding agent specifically binds to the first molecule and the second primary binding agent specifically binds to the second molecule; and contacting the sample with a first hybrid, which comprises a first binding partner and a first nucleic acid, and a second hybrid, which comprises a second binding partner and a second nucleic acid. The first binding partner specifically binds to the first primary binding agent and the second binding partner specifically binds to the second primary binding agent. The first nucleic acid comprises a first nucleotide sequence complementary to a second nucleotide sequence in the second nucleic acid and is capable of forming a hybridization product with the second nucleic acid when the first hybrid is bound to the first primary binding agent and the second hybrid is bound to the second primary binding agent and the first primary binding agent and the second primary binding agent are in proximity. The hybridization product optionally is extended to form an extension product, and the presence or absence of the hybridization product or the extension product is identified, whereby the presence of the hybridization product or the extension product detects the first molecule and the second molecule in the sample. This method is applicable to detecting a molecule in a sample where the first primary binding agent and the second primary binding agent bind to binding regions in the molecule. Many of the embodiments described herein are applicable to detecting a molecule or molecules with secondary MAD reagents. In certain embodiments the first primary binding agent is an antibody or antibody fragment from a first animal and the second primary binding agent is an antibody or antibody fragment from a second animal. In some embodiments, the first primary binding agent is an antibody or antibody fragment of a first isotype and the second primary binding agent is an antibody or antibody fragment of a second isotype. In some embodiments, the first and the second primary binding agent are monoclonal antibodies of a first subtype from the same species, and the secondary MAD hybrids bind to antibodies or antibody fragments of the first subtype from the species. MAD multiplex assays and reagents

MAD assays sometimes are utilized for detecting multiple molecular antigens in a single sample in a process referred to herein as “multiplexing.” Multiplexing is applicable to many embodiments described herein. A hybrid used in multiplex assays sometimes specifically hybridizes to one other hybrid added to the system, and sometimes hybridizes to two or more distinct hybrids added to the system. MAD hybrid nucleic acids used for multiplexing applications often include a hybridization subsequence that hybridizes to a nucleic acid of another hybrid, and include a unique nucleotide subsequence. The hybridization subsequence in a hybrid sometimes is selected to hybridize to one other hybrid in a multiplex assay, and sometimes is selected to hybridize to two or more other hybrids in the multiplex assay. The unique subsequence is identified in a hybridization product or extension product, sometimes by an amplification process (e.g., PCR) and sometimes by a nucleic acid binding agent that specifically binds to the unique subsequence. Where amplification is utilized for detection, amplification products having different multiplexing nucleotide sequences are detected and resolved by an appropriate method. In an embodiment, the amplification products are resolved and detected according to size by gel electrophoresis (e.g., plate or capillary gels of polyacrylamide or agarose). In some embodiments, the amplification products are resolved and detected according to sequence by hybridizing them to another oligonucleotide or set of oligonucleotides (e.g., performing real time quantitative PCR (RT-PCR) procedures using TaqMan® (Applied Biosystems, Inc.) or LUX™ (Invitrogen, Inc.) systems).

Thus, provided herein is a method for detecting multiple proximal molecules in a sample, which comprises contacting a sample with a first hybrid comprising a first binding partner and a first nucleic acid, a second hybrid comprising a second binding partner and a second nucleic acid, and a third hybrid comprising a third binding partner and a third nucleic acid. The first binding partner specifically binds to a first molecule, the second binding partner specifically binds to a second molecule, and the third binding partner specifically binds to a third molecule. The first nucleic acid comprises a first nucleotide sequence complementary to a second nucleotide sequence in the second nucleic acid and is capable of forming a hybridization product with the second nucleic acid when the first hybrid is bound to the first molecule and the second hybrid is bound to the second molecule and the first molecule and the second molecule are in proximity in the sample. The first nucleotide sequence also is complementary to a third nucleotide sequence in the third nucleic acid, the first nucleic acid is capable of forming a hybridization product with the third nucleic acid when the first hybrid is bound to the first molecule and the third hybrid is bound to the third molecule and the first molecule and the third molecule are in proximity in the sample. Hybridization products often are extended and hybridization products or extension products are detected using agents that specifically identify hybridization products formed by the first and second hybrids and formed by the first and third hybrids. While these embodiments specify that the sample is contacted with three hybrids, MAD assays sometimes are performed using four or more hybrids, where all of the hybrids include a unique multiplexing nucleotide sequence.

The second nucleic acid often comprises a fourth nucleotide sequence in a non-overlapping region of the hybridization product between the first nucleic acid and the second nucleic acid, and the third nucleic acid often comprises a fifth nucleotide sequence in a non-overlapping region of the hybridization product between the first nucleic acid and the third nucleic acid. The fourth nucleotide sequence is not identical to a nucleotide sequence in the first nucleic acid or third nucleic acid and the fifth nucleotide sequence is not identical to a nucleotide sequence in the first nucleic acid or second nucleic acid. Hybridization products between the first nucleic acid and the second nucleic acid and between the first nucleic acid and the third nucleic acid, or extension products generated therefrom, sometimes are detected by contacting the sample with PCR reagents (e.g., a polymerase, nucleotides, and one or more oligonucleotides) that amplify the fourth nucleotide sequence, the fifth nucleotide sequence, or the fourth nucleotide sequence and the fifth nucleotide sequence, and detecting amplification products. The presence of an amplification product containing the fourth nucleotide sequence is indicative of the first molecule and the second molecule being in proximity in the sample, and detecting an amplification product containing the fifth nucleotide sequence is indicative of the first molecule and the third molecule being in proximity in the sample.

Two or more reporter molecules sometimes are utilized in multiplex MAD assays to detect multiple molecular antigens in a sample. In an embodiment, a sample is contacted with a first hybrid, a second hybrid, and a third hybrid, where the nucleic acids of the first hybrid and second hybrid are capable of forming a hybridization product when a first molecule and a second molecule to which the hybrids bind are in proximity in the sample, and where nucleic acids of the first hybrid and the third hybrid are capable of forming a hybridization product when the first molecule and a third molecule to which the hybrids bind are in proximity in the sample. In this embodiment, the region to which the nucleic acid binding protein in each reporter molecule binds is in the nucleic acids linked to the second and third hybrids.

In other embodiments, a sample is contacted with a first hybrid, a second hybrid, a third hybrid, and a fourth hybrid, where the first hybrid binds to a first molecule, the second hybrid binds to a second molecule, the third hybrid binds to a third molecule, and the fourth hybrid binds to a fourth molecule. The nucleic acids of the first hybrid and second hybrid are capable of forming a hybridization product when the first molecule and a second molecule are in proximity in the sample, and where nucleic acids of the third hybrid and the fourth hybrid are capable of forming a hybridization product when the third molecule and fourth molecule are in proximity in the sample. In an alternative embodiment, the fourth hybrid binds to the first molecule and includes a nucleic acid that differs from the first hybrid, where a hybridization product is capable of forming between nucleic acids of the first and second hybrids when the first and second molecules are in proximity and another hybridization product is capable of forming between nucleic acids of the third and fourth hybrids when the first and third molecules are in proximity. In the second embodiment, the binding region to which a nucleic acid binding protein in a reporter molecule binds spans overlapping and non-overlapping regions of the hybridization products formed between the hybrids. In these embodiments, each hybridization product optionally is extended by contacting the hybridization product with a polymerase and nucleotides. The sample then is contacted with a first reporter molecule and a second reporter molecule, where the first reporter molecule specifically binds to the hybridization product and/or the extension product formed by nucleic acids of the first hybrid and the second hybrid, and where the second reporter molecule specifically binds to the hybridization product and/or the extension product formed by the nucleic acids of the third hybrid and forth hybrid. The first reporter molecule is linked to a detectable label different than the detectable label linked to the second reporter molecule and signals from the labels are detected and resolved. In a specific embodiment, the first reporter molecule includes a fluorescein label and the second reporter molecule includes a rhodamine molecule. Different excitation wavelengths and different emission wavelengths are utilized to detect fluorescein and rhodamine and accordingly, the first reporter molecule and the second reporter molecule are distinguished in a sample. In some embodiments, the first reporter molecule includes a green fluorescent protein (GFP), yellow fluorescent protein (YFP) or blue fluorescent protein (BFP) and the second reporter molecule includes one of these proteins which is not linked to the first reporter molecule. Signal generated form each reporter molecule sometimes is quantified utilizing standard techniques, which often include comparing the signal generated in a sample to the signal generated by a control sample having known quantities of reporter molecule and/or a control sample having no reporter molecule. As described herein, two or more reporter molecules also can be utilized in FRET assay embodiments.

Immobilization of MAD Assay Components to a Solid Support

MAD assay components include hybrids, molecules comprising antigens to which the hybrids bind, cellular complex components in association with the molecules to which the hybrids are bound, hybridization products and extension products. One or more of these components sometimes are immobilized to a solid support or solid phase at any time in the MAD assay. MAD assay components often are immobilized by non-covalent interactions, and sometimes are linked by covalent interactions. Appropriate solid supports are utilized, which include a microtiter plate well surface, a silicon wafer surface, the surface of a bead (e.g., a magnetic bead) or a channel in a microfluidic device. Solid supports, linking molecules for covalent and non-covalent attachments to the solid supports, and methods for immobilizing nucleic acids and other molecules to solid supports are known (see e.g., U.S. Pat. Nos. 6,261,776; 5,900,481; 6,133,436 and 6,022,688; and WO 01/18234).

MAD assay components sometimes are passively immobilized to a solid phase. In embodiments where a hybrid binding partner is an antibody, the hybrid can be immobilized to a solid phase pre-treated with an Fc receptor or S. aureus, both of which non-covalently bind the hybrid antibody. In certain embodiments, the solid support is derivitized with a protein that binds to a MAD assay component (e.g., an antibody or a nucleic acid binding agent). Nucleic acid binding proteins described previously often are utilized to immobilize MAD assay components to a solid support. In an embodiment, a lac repressor protein or a functional fragment or sequence variant thereof, which specifically binds to a nucleotide sequence within a hybridization product or in an extension product generated from the hybridization product, is joined to a solid support before, during, or after the nucleic acid binding protein is contacted with the MAD assay components. Several strategies are available for joining the nucleic acid binding protein to a solid support. In an embodiment, the nucleic acid binding protein is linked to a first linking molecule and is joined to a solid support derivitized with a second linking molecule that specifically binds to the first linking molecule. The solid support sometimes is a resin, and sometimes the resin is in a column and is utilized in chromatography methods. The first linking molecule sometimes is biotin, avidin or streptavidin. In certain embodiments, the solid support is derivitized with an antibody that specifically binds to the nucleic acid binding protein, where the nucleic acid binding protein is capable of binding the hybridization product or extension product when bound to the antibody joined to the solid support.

In some embodiments, MAD assay components are joined to a solid support derivitized with a first linking molecule that specifically binds to a second linking molecule on a MAD assay component. In an embodiment, a hybrid is derivitized with a first linking moiety (e.g., biotin, avidin or streptavidin) and contacted with a solid phase comprising a second linking moiety that specifically binds to the first linking moiety. In certain embodiments, a biotin-derivitized extension product is generated by contacting a hybridization product with a polymerase and one or more biotinylated nucleotides, and contacting the biotinylated MAD assay component with a solid support derivitized with avidin or streptavidin.

Methods for conjugating linking molecules such as avidin, streptavidin and biotin to other molecules are known (e.g., U.S. Pat. Nos. 6,214,974 (Rosenblum, et al.) and 6,429,297 (Rosebrough)). For example, avidin sometimes is conjugated to a molecule by derivitizing the molecule with N-succinimidyl 3-(2-pyridyldithio)propionate (SPDP), derivitizing avidin with 2-iminothiolane, and contacting the SPDP-derivitized molecule and 2-iminothiolane-modified avidin with one another in the presence of iodoacetamide. Biotin sometimes is conjugated to a molecule by contacting N-hydroxy succinimide ester long chain (NHS-LC) biotin with the target molecule in dimethylformamide. An antibody or nucleic acid binding protein sometimes is linked to a solid support by generating a maleimide-derivitized antibody or nucleic acid binding protein, contacting it with an avidin molecule having a sulfhydryl moiety and then linking the conjugate to a commercially available biotinylated solid support (see, e.g., U.S. Pat. No. 6,429,297 (Rosebrough)).

Solid phase approaches are applicable to many of the embodiments described herein. For example, provided herein is a method for detecting a first molecule and a second molecule in a sample, which comprises contacting a sample with a first hybrid in association with a solid phase, where the first hybrid comprises a first binding partner and a first nucleic acid. The first hybrid and sample are contacted with a second hybrid, where the second hybrid comprises a second binding partner and a second nucleic acid. The solid phase sometimes is washed after it is contacted with the sample and before it is contacted with the second hybrid and sometimes it is washed after it is contacted with the second hybrid. The second hybrid and sample sometimes are added to the solid phase at the same time. The first binding partner specifically binds to the first molecule and the second binding partner specifically binds to the second molecule. The first nucleic acid comprises a first nucleotide sequence complementary to a second nucleotide sequence in the second nucleic acid and is capable of forming a hybridization product with the second nucleic acid when the first hybrid is bound to the first molecule and the second hybrid is bound to the second molecule and the first molecule and the second molecule are in proximity. The hybridization product sometimes is extended to form an extension product, and the presence or absence of the hybridization product or the extension product is identified, whereby the presence of the hybridization product or the extension product detects the first molecule and the second molecule in the sample. This method is applicable to detecting a molecule in a sample where the first hybrid and the second hybrid bind to binding regions in the molecule.

MAD assay components often are treated after they are immobilized to a solid support. In an embodiment, MAD assay components immobilized to a solid support are separated from components of a sample not immobilized to the solid support. In certain embodiments, immobilized MAD assay components are detected by a suitable method, such as PCR processes or reporter molecule processes described previously. In some heterogeneous assay embodiments, the solid phase is washed to remove components that give rise to background signals.

In some embodiments, immobilized MAD assay components are released from the solid support and separated from one another, where some or all of the MAD assay components are released from the solid support. Any suitable method for releasing molecules from the solid support is utilized, such as denaturing protein components with a detergent (e.g., sodium dodecyl sulfate) or by contacting the solid support with an acid, for example. As some of the molecules released from the solid support sometimes are non-antigen proteins or non-hybrid nucleic acids in association with antigen molecules in the sample (e.g., non-antigen proteins in the same complex as the antigen molecules in the sample), such methods are useful for detecting non-antigen components in proximity to the antigen molecules or in a complex with the antigen molecules in the sample or cells from which the sample is prepared. Components released from the solid support often are subjected to further separation and characterization techniques, such as denaturing or non-denaturing gel electrophoresis, antibody binding, Western blotting, mass spectrometry, purification, use as an antigen for generating antibodies, nucleotide sequencing and amino acid sequencing, for example.

MAD assay components sometimes are in association with one or more non-hybrid nucleic acids. The non-hybrid nucleic acid sometimes is a fragment of cellular chromatin DNA, sometimes a nucleic acid from a DNA library (e.g., cDNA or a genomic DNA library), and sometimes an RNA molecule. The non-hybrid nucleic acid sometimes is released from the solid support by a suitable technique (e.g., treating the solid support with an enzyme that digests proteins (e.g., pronase)). One or more non-hybrid nucleic acid fragments sometimes are in association with MAD assay components, especially when nucleic acids are fragmented in the sample (e.g., MAD-ChIP assays). A non-hybrid nucleic acid often is subjected to further separation, amplification and/or analysis (e.g., gel electrophoresis and/or nucleotide sequencing). A nucleotide sequence of the non-hybrid nucleic acid sometimes is deduced by standard recombinant techniques, such as by cloning a non-hybrid nucleic acid or a portion thereof into a plasmid designed for nucleotide sequencing (e.g., conventional cloning techniques or TOPO® cloning vectors, Invitrogen, Inc.), amplifying the cloned plasmid, and sequencing the non-hybrid nucleic acid portion of the plasmid (e.g., using an ABI 377 automated sequencer (Applied Biosystems)), for example. Conventional or TOPO® cloning techniques are useful for recovering a desired sequence or for generating a library of all non-hybrid sequences bound to the solid support. These embodiments are useful for determining a nucleotide sequence of a non-hybrid nucleic acid component, which sometimes is associated with an antigen to which a MAD hybrid binds or to a protein in the same complex as an antigen to which a MAD assay hybrid binds. The non-hybrid nucleic acid sometimes is from a nucleic acid library. The non-hybrid nucleic acid often is genomic DNA from a cell (i.e., chromatin DNA), or processed from genomic DNA, which is described in greater detail hereafter.

MAD-ChIP Assays and Reagents

MAD chromatin immunoprecipitation (MAD-ChIP) assays are featured herein. In general, MAD-ChIP assays are performed by cross-linking molecules, fragmenting chromatin, and detecting molecules in association with chromatin using MAD hybrids and detection methods described herein. One embodiment is a method for determining whether a first molecule and a second molecule are in proximity in a sample, which comprises contacting a cell or organism or a sample prepared therefrom with a cross-linking agent. The sample is contacted with a first hybrid and a second hybrid, where the first hybrid comprises a first binding partner and a first nucleic acid and the second hybrid comprises a second binding partner and a second nucleic acid. The first binding partner specifically binds to the first molecule and the second binding partner specifically binds to the second molecule. The first nucleic acid comprises a first nucleotide sequence complementary to a second nucleotide sequence in the second nucleic acid and is capable of forming a hybridization product with the second nucleic acid when the first hybrid is bound to the first molecule and the second hybrid is bound to the second molecule and the first molecule and the second molecule are in proximity. The hybridization product optionally is extended and the presence or absence of the hybridization product or extension product is identified, whereby the presence of the hybridization product is indicative of the first molecule and the second molecule being in proximity. In particular embodiments, the first molecule and second molecule are independently selected from a protein or chromatin DNA (sometimes referred to as “non-hybrid DNA”); the first molecule and the second molecule are proteins in association with chromatin DNA; and the first molecule is a protein and the second molecule is a non-hybrid DNA, where the non-hybrid DNA is chromatin or is derived from chromatin. In a specific embodiment, multiplexing procedures described previously are applied to MAD-ChIP assays, where three or more molecules are detected in the MAD-ChIP assays.

In certain embodiments, the chromatin DNA probed by MAD-ChIP assays is genomic DNA isolated directly from an organism without genetic modification; DNA from a recombinant library; or chemically altered DNA (e.g., cells are treated with bromodeoxyuridine (BrdU) or a related nucleotide analog, which incorporates into the genomic DNA of dividing cells). In some embodiments, the chromatin DNA is genetically modified by standard recombinant DNA techniques, and in particular embodiments, one or more heterologous or exogenous nucleotide sequences are inserted into the genomic DNA by standard techniques. The heterologous or exogenous nucleotide sequence often is a sequence from genomic DNA that regulates replication or transcription of a proximal gene (e.g., an origin recognition sequence or a promoter), and sometimes the heterologous or exogenous nucleotide sequence is from a recombinant DNA library. In certain embodiments, one hybrid binds to a protein bound to the heterologous or exogenous nucleotide sequence and another hybrid binds to a proximal antigen, where the proximal antigen, for example, comprises or consists of double stranded DNA, a BrdU molecule in labeled DNA or a portion of a histone. In other embodiments, the heterologous or exogenous nucleotide sequence binds to a nucleic acid binding protein, and sometimes is oriented in proximity to another exogenous or heterologous regulatory sequence and consequently acts as a “homing” sequence for MAD assay components (e.g., one hybrid binds to the nucleic acid binding protein bound to the homing sequence and another hybrid binds to a protein bound to the exogenous or heterologous nucleotide sequence). The homing sequence sometimes is designed to insert adjacent to a regulatory region already present in genomic DNA of a cell, and sometimes is adjacent to a regulatory region in a nucleic acid and a homing sequence/regulatory region sequence is inserted into genomic DNA of a cell.

In an embodiment, the homing sequence comprises or consists of a regulatory sequence such as a lac operon (e.g., lacO) sequence, to which binds a lac repressor protein, a substantially identical protein, or a functional fragment thereof. LacO has the sequence AATTGTGAGCGGATAACAATT, which binds the lac repressor with high affinity (K_{DNA}=2 to 5×10¹³ M⁻¹) and multiple known lacO sequences bind the repressor with higher or lower affinity (e.g., Barkley &. Bourgeois, In The Operon Miller & Reznikoff, eds. Cold Spring Harbor (1978); U.S. Pat. No. 5,169,760 (Wilcox)). In other embodiments, the homing sequence is a nucleotide sequence to which a lexA protein binds (e.g., the lexA protein binds to short binding sites in E. coli genetic control regions or operators (e.g., Brent & Ptashne, Proc Natl Acad Sci U.S.A. 78(7): 4204-8 (1981)and Little & Mount, Cell 29(1):11-22 (1982))); a sequence to which a gal4 protein binds (e.g., the gal4 protein binds to DNA at short binding sites within upstream activating sequences (UASs) in the yeast (S. cerevisiae) genome (e.g., Giniger et al., Cell. 40(4): 767-74 (1985); Bram et al., EMBO J 5(3): 603-8 (1986) and Ptashne, Nature 20; 335(6192): 683-9 (1988))); or a ter nucleotide sequence to which a tus protein binds (e.g., the tus protein binds DNA at ter sites within the replication terminus of the E. coli genome (Sista et al., Proc Natl Acad Sci U.S.A. 86(9): 3026-30 (1989); Hill et al., Proc Natl Acad Sci U.S.A. 86(5):1593-7 (1989) and Kuempel, Cell 17; 59(4): 581-3 (1989))). Methods are known for introducing one or more heterologous or exogenous nucleotide sequences into genomic DNA. In certain embodiments, an exogenous or heterologous nucleotide sequence is introduced into genomic DNA by recombinase-mediated cassette exchange. (See, e.g., Feng, et al., J. Mol. Biol. 292:779-785 (1999); Scibler and Bode, Biochemistry 36:1740-1747 (1997); Baer & Bode, Curr. Opin. Biotechnol. 12:473-480 (2001); and Belteki, et al., Nat. Biotechnol. 21:321-324 (2002)).

Molecules are cross-linked using an appropriate chemical linker that yields a reversible or non-reversible linkage (see e.g., Orlando, et al., Methods 11:205-214 (1997)). In an embodiment, formaldehyde is utilized as a reversible cross-linking agent (see e.g., Johnson & Bresnick, Methods 26:27-36 (2002)). The cross-linking agent often is contacted with an organism or a cell (e.g., a non-disrupted cell) and sometimes is contacted with a cell lysate. In an embodiment, a cell is contacted with a cross linking agent and the cell then is lysed. Cells often are exposed to certain molecules or conditions described previously (e.g., a small organic or inorganic molecule or ionizing radiation), before being exposed to a cross-linking agent. Cross-linking agents frequently link adjacent molecules to one another in a cell or sample, such that molecular antigens in a sample sometimes are directly cross-linked with one another, and sometimes are indirectly cross-linked to one another where one or more non-antigen molecules intervene. Thus, molecular antigens often are cross-linked to non-antigen proteins and non-hybrid nucleic acids, and sometimes are linked to another molecular antigen.

After a cross-linked sample is prepared, cross-linked chromatin DNA often is fragmented using an appropriate process, such as sonication or shearing through a needle and syringe, for example. Using sonication, chromatin fragments of about 500 to about 1000 base pairs in length often are obtained. In some embodiments, cross-linked chromatin is separated from other sample components before fragmentation, and in other embodiments fragmented chromatin is separated from other assay components before the chromatin fragments are contacted with MAD hybrids, as described hereafter. Cross-linked chromatin or chromatin fragments are separated from other sample components by an appropriate process, such as density centrifugation, gel electrophoresis or chromatography, for example.

Chromatin (e.g., cross-linked chromatin, fragmented chromatin, or cross-linked and fragmented chromatin) is contacted with MAD hybrids to detect molecular antigens in association with the chromatin. Conditions in which the chromatin is contacted with the MAD hybrids are the same or similar to those described previously. MAD hybridization products and/or extension products are directly or indirectly detected using the methods described previously. In an embodiment, a hybridization product is formed between complementary regions of hybrid nucleic acids, the hybrid is extended, and the extension product is detected by PCR or by a nucleic acid binding protein linked to a detectable label.

In certain embodiments, a hybridization product is formed between complementary hybrid nucleic acids, the hybridization product is extended, and the extension product is contacted with a nucleic acid binding protein that binds to a nucleic sequence in the extension product, where the nucleic acid binding protein is linked to a solid support before, during, or after it is contacted with the extension product. In other embodiments, a hybridization product is formed between complementary hybrid nucleic acids, the hybridization product is extended using nucleotides that comprise a first linking molecule (e.g., biotin), and the extension product is contacted with a solid support derivitized with a second linking molecule complementary to the first linking molecule in the extension product (e.g., avidin) such that the solid support immobilizes the extension product. In the latter two embodiments which involve immobilizing an extension product to a solid support, subsequent steps sometimes are performed. For example, the immobilized extension product sometimes is treated with an agent that digests proteins in association with the extension product (e.g., a protease such as pronase that digests antigen proteins and binding partner proteins such as antibodies), leaving the extended hybrid nucleic acids and chromatin fragments previously bound to or in association with the hybrids. In some embodiments, cross-linking is reversed using standard techniques (e.g., heating the sample) and extension product components are separated from one another as described previously. In certain embodiments, one or more chromatin DNA fragments that were cross-linked or in association with MAD assay components (e.g., antigen or non-antigen proteins) are sequenced using standard techniques (e.g., using a TOPO® cloning plasmid) after subsequent steps are performed.

In embodiments where the cellular genomic DNA is modified by recombinant procedures to include a homing sequence to which its cognate binding partner binds, the cognate binding protein sometimes is the first molecule and the first binding partner in the MAD hybrid sometimes is an antibody that binds to the cognate binding protein. In an example of such embodiments, the lac repressor protein is the first molecule and the first binding partner in the MAD hybrid is an antibody that specifically binds to the lac repressor protein. In embodiments in which the genomic DNA is modified to include an origin recognition regulatory region, the second molecule sometimes is an origin recognition protein (ORC) and the second binding partner is an antibody that specifically binds to the ORC protein. In some embodiments, the first binding partner in the first hybrid is a lacO binding protein (e.g., lac repressor) linked to single stranded DNA.

In some embodiments, the first molecule is double-stranded DNA or a bromodeoxyuridine (BrdU) molecule in association with double-stranded DNA, and the first binding partner is an antibody that specifically binds to the double-stranded DNA or the BrdU molecule. In a related embodiment, the second molecule is p53 and the second binding partner is an antibody that specifically binds to p53. In an alternative embodiment, the second molecule is an ORC protein and the second binding partner is an antibody that specifically binds to the ORC protein. In other embodiments, one of the molecules is C-MYC and the binding partner is an antibody which specifically binds to an antigen in C-MYC; one of the molecules is beta-catenin/TCF and the binding partner is an antibody that specifically binds to an antigen in beta-catenin/TCF; and one of the molecules is fos-jun and the binding partner is an antibody which specifically binds to an antigen infos-jun.

In certain embodiments, chromatin DNA in association with a MAD assay component is sequenced using procedures described herein. In other embodiments, samples are exposed to different conditions or exogenous molecules, and molecules detected by MAD-ChIP assays as being in proximity with one another are compared for each condition or exogenous molecule. These embodiments are useful for determining which molecules exist in a chromatin complex when samples are exposed to different conditions or exogenous molecules. For example, it can be determined which proteins are in association with a particular chromatin DNA under various conditions. It also can be determined which nucleic acids are in association with a particular protein under various conditions, which involves sequencing nucleic acids in association with MAD assay components in the MAD-ChIP assays.

Thus, provided is a method for determining the nucleotide sequence of one or more genomic DNA fragments in association with a molecule of interest, which comprises contacting a sample comprising a genomic DNA fragment with a first hybrid and a second hybrid, where the first hybrid comprises a first binding partner and a first nucleic acid and the second hybrid comprises a second binding partner and a second nucleic acid. The first binding partner specifically binds to the molecule of interest and the second binding partner binds to double-stranded DNA. The first nucleic acid comprises a first nucleotide sequence complementary to a second nucleotide sequence in the second nucleic acid and is capable of forming a hybridization product with the second nucleic acid when the first hybrid is bound to the molecule of interest and the second hybrid is bound to double-stranded DNA and the molecule of interest and the genomic DNA are in proximity. The hybridization product sometimes is extended to form an extension product. The hybridization product or the extension product and a genomic DNA fragment in contact with it are isolated, and the nucleotide sequence of the genomic DNA fragment is identified.

Also provided is a method for determining the nucleotide sequence of one or more genomic DNA fragments in association with two molecules of interest, which comprises contacting a sample comprising a genomic DNA fragment with a first hybrid and a second hybrid, where the first hybrid comprises a first binding partner and a first nucleic acid and the second hybrid comprises a second binding partner and a second nucleic acid. The first binding partner specifically binds to the first molecule of interest and the second binding partner binds to the second molecule of interest. The first nucleic acid comprises a first nucleotide sequence complementary to a second nucleotide sequence in the second nucleic acid and is capable of forming a hybridization product with the second nucleic acid when the first hybrid is bound to the first molecule of interest and the second hybrid is bound to the second molecule of interest and the first and second molecules of interest are in proximity when in association with genomic DNA. The hybridization product sometimes is extended to form an extension product in some embodiments. The hybridization product or the extension product and a genomic DNA fragment in contact with it are isolated, and the nucleotide sequence of the genomic DNA fragment is determined.

Provided also is a method for identifying a molecule of interest in association with a nucleic acid regulatory region, which comprises inserting the nucleic acid regulatory region into genomic DNA of a cell and contacting a sample comprising the cell or prepared from the cell with a first hybrid and a second hybrid, where the first hybrid comprises a first binding partner and a first nucleic acid and the second hybrid comprises a second binding partner and a second nucleic acid. The first binding partner specifically binds to the molecule of interest and the second binding partner binds to another component in the cell in association with the regulatory region. The first nucleic acid comprises a first nucleotide sequence complementary to a second nucleotide sequence in the second nucleic acid and is capable of forming a hybridization product with the second nucleic acid when the first hybrid is bound to the molecule of interest and the second hybrid is bound to the other component in the cell and the molecule of interest and the other component are in proximity. The hybridization product sometimes is extended to form an extension product and the presence or absence of the hybridization product or the extension product is identified, whereby the presence of the hybridization product or the extension product is indicative of the molecule of interest being in association with the nucleic acid regulatory sequence. In some embodiments, the other component to which the second binding partner and second hybrid specifically binds is selected from the group consisting of DNA, a histone, a protein in proximity to the molecule of interest, and a modified nucleotide moiety in DNA.

Also provided is a method for identifying a molecule of interest in association with a nucleic acid regulatory region, which comprises inserting a nucleic acid comprising a homing sequence adjacent to the regulatory region in genomic DNA of a cell or inserting a nucleic acid comprising the regulatory region adjacent to a homing sequence into genomic DNA of a cell, and contacting a sample comprising the cell or prepared from the cell with a first hybrid and a second hybrid, where the first hybrid comprises a first binding partner and a first nucleic acid and the second hybrid comprises a second binding partner and a second nucleic acid. The first binding partner specifically binds to the molecule of interest and the second binding partner binds to a molecule in association with the homing sequence. The first nucleic acid comprises a first nucleotide sequence complementary to a second nucleotide sequence in the second nucleic acid and is capable of forming a hybridization product with the second nucleic acid when the first hybrid is bound to the molecule of interest and the second hybrid is bound to the molecule in association with the homing sequence and the molecule of interest and the molecule in association with the homing sequence are in proximity. The hybridization product sometimes is extended to form an extension product and the presence or absence of the hybridization product or the extension product is identified, whereby the hybridization product or the extension product is indicative of the molecule of interest being in association with the nucleic acid regulatory sequence. In some embodiments, the homing sequence is a lacO sequence and the molecule in association with the homing sequence is identical to or substantially identical to a lac repressor protein or a functional fragment thereof.

Many of the embodiments described herein are applicable to methods for determining a nucleotide sequence of a nucleic acid associated with a target molecule, methods for identifying a molecule of interest in association with a nucleic acid regulatory region and methods for identifying a molecule of interest in association with a nucleic acid regulatory region. For example, one or more nucleotides used to form the extension product sometimes are biotinylated and the biotinylated extension product is isolated by contacting it with avidin, where the avidin is immobilized to a solid support before, during or after the avidin is contacted with the biotinylated extension product. In some embodiments, the hybridization product or the extension product is isolated by contacting it with a nucleic acid binding agent, where the nucleic acid binding agent is linked to a solid support before, during, or after the nucleic acid binding agent is contacted with the hybridization product or the extension product. The nucleic acid binding agent often is a protein that is identical to or substantially identical to a lac repressor protein or a functional fragment thereof. In some embodiments, the sample is contacted with a cross-linking agent. In certain embodiments, the process further comprises removing protein bound to the genomic DNA fragment.

In Situ Cellular MAD Assays

MAD assays are applicable to determining whether molecules are in proximity in a cell or on a cell surface. Thus, provided herein is a method for detecting a first molecule and a second molecule in a cell, which comprises contacting the cell with a first hybrid and a second hybrid, where the first hybrid comprises a first binding partner and a first nucleic acid and the second hybrid comprises a second binding partner and a second nucleic acid. The first binding partner specifically binds to the first molecule and the second binding partner specifically binds to the second molecule. The first nucleic acid comprises a first nucleotide sequence complementary to a second nucleotide sequence in the second nucleic acid and is capable of forming a hybridization product with the second nucleic acid when the first hybrid is bound to the first molecule and the second hybrid is bound to the second molecule and the first molecule and the second molecule are in proximity. The hybridization product optionally is extended to form a hybridization product, and the presence or absence of the hybridization product or the extension product is identified, whereby the hybridization product or the extension product is indicative of the first molecule and the second molecule being in proximity. A related method is detecting a molecule in a cell, where the first hybrid and the second hybrid specifically bind to binding regions in the molecule.

Molecular antigens can be detected in any type of cell or group of cells, including bacteria (e.g., E. coli); yeast; fungi; slime mold; invertebrate cells (e.g., insect (e.g., Drosophila), nematode and flatworm cells) and vertebrate cells (e.g., fish (e.g., zebra fish), amphibian cells (e.g., frogs (e.g., Xenopus)), avian cells, and mammalian cells (e.g., hamster, guinea pig, mouse, rat, rabbit, canine, feline and primate (e.g., chimpanzee, baboon, or human)). Cell samples can be prepared using any of the techniques described herein (e.g., the cells are fixed, permeablized and/or lysed), and in specific embodiments, the cells are living (e.g., dividing, proliferating and/or growing in size) or are not living (e.g., not dividing, proliferating or growing in size). For assay embodiments involving living cells, cells often are microinjected with hybrids and other MAD assay components and then hybridization products or extension products are detected. Cells often are mounted onto a solid support, sometimes are contacted with a cross-linking agent and sometimes are made permeable (Melan, Methods Mol. Biol. 34:55-66 (1994)). Cells sometimes are washed after being contacted with hybrids to remove unbound hybrid (e.g., especially when hybrids designed not to form hybridization products are utilized), and hybrid nucleic acids, hybridization products, or extension products then are detected.

Many of the embodiments described herein are applicable to methods for detecting a molecule or molecules in a cell. For example, the hybridization product is extended in some embodiments, and sometimes the amount of hybridization product or extension product is quantified. Nucleic acid binding agents often are utilized as reporter molecules to detect MAD assay components in situ, and such agents can be utilized to detect multiple molecules in a cell using the multiplexing procedures described above. In an embodiment, one reporter molecule comprises a nucleic acid binding protein identical to or substantially identical to a lac repressor protein or fragment thereof, which binds to a functional lacO subsequence in a hybrid nucleic acid, hybridization product or extension product. The nucleic acid binding agent often comprises a detectable label, including but not limited to a fluorescent label, light-emitting label or light-scattering label. In some embodiments, the reporter molecule is a fusion protein (e.g., lac repressor protein linked to a green fluorescent protein (GFP), yellow fluorescent protein (YFP), or blue fluorescent protein (BFP)), and other embodiments involve indirect detection (e.g., using a FITC-labeled anti-lac repressor antibody). The hybridization product or extension product sometimes are linked to a solid-phase, and in some embodiments, a nucleic acid binding agent added to the system binds the hybridization product or extension product and is linked to a solid support before, during or after it is added to the system. In some embodiments, the extension product includes one or more nucleotides which comprise a first linking partner capable of binding to a second linking partner, and the second linking partner is joined to a solid support before, during, or after the first linking partner is contacted with the second linking partner. The first linking partner and second linking partners are independently selected from linking moieties described herein, such as biotin, avidin and streptavidin. In some embodiments, components in the hybridization complex are separated from one another, such as the first molecule, second molecule, first hybrid, second hybrid and any non-hybrid nucleic acids, and sometimes the nucleotide sequence of a non-hybrid nucleic acid in association with a hybrid is determined.

Reporter molecules often are detected in situ (e.g., in cells and/or on cell surfaces) using known techniques. Such techniques include using a standard light microscope or a confocal microscope (e.g., U.S. Pat. Nos. 5,283,433 and 5,296,703 (Tsien)). Appropriate light microscopes are commercially available and are useful for probing cells in two dimensions (i.e., the height of a cell often is not resolved), and confocal microscopy is useful for probing cells in three dimensions. Many microscopy techniques are useful for determining the location of molecular antigens in a cell (e.g., in the nucleus, cytoplasm, plasma cell membrane, nucleolus, mitochondria, vacuoles, endoplasmic reticulum or Golgi apparatus). Some microscopic techniques are useful for determining the location of molecular antigens in groups of cells, tissue samples, and organs. Cellular locations often are visualized by counter-staining for subcellular organelles.

Many of the embodiments described herein are applicable to the methods described in this section. In certain embodiments, cells are exposed to different conditions or exogenous molecules, and molecules detected by MAD assays as being in proximity with one another in the cells are compared between each condition or exogenous molecule. These embodiments are useful for determining which molecules exist in a complex when cells are exposed to different conditions or exogenous molecules. For example, it can be determined which proteins are differentially in association with a particular genomic DNA sequence in cells under various conditions, and it can be determined which nucleic acids are differentially in association with a particular protein under various conditions. In other embodiments, it can be determined to which cellular regions (e.g., nucleus, membrane, cytoplasm, specific organelle) certain proteins or nucleic acid sequences are co-localized when cells are exposed to specific conditions or exogenous molecules.

MAD Assays in Which a Sample is Exposed to a Specific Condition

The sample sometimes is exposed to a certain condition before the sample is contacted with a hybrid, and at times the sample is contacted with a hybrid before or at the same time it is exposed to the condition. MAD assay results generated from samples exposed to different conditions often are compared to one another, and MAD assay results generated from samples exposed to a particular condition often are compared to MAD assay results generated from samples not exposed to the same condition. For example, particular protein molecules or nucleic acid molecules identified by MAD assays as being in proximity for one set of conditions often are compared to particular protein molecules or nucleic acid molecules identified by MAD assays as being in proximity for another set of conditions. This type of analysis can determine which molecules are in proximity or in a complex in a sample under specific conditions.

Thus, provided herein is a method for determining whether a first molecule and a second molecule are in proximity in a sample under a specific condition, which comprises exposing a sample to a condition; contacting the sample with a first hybrid and a second hybrid, where the first hybrid comprises a first binding partner and a first nucleic acid and the second hybrid comprises a second binding partner and a second nucleic acid, where the first binding partner specifically binds to the first molecule and the second binding partner specifically binds to the second molecule. The first nucleic acid comprises a first nucleotide sequence complementary to a second nucleotide sequence in the second nucleic acid and is capable of forming a hybridization product with the second nucleic acid when the first hybrid is bound to the first molecule and the second hybrid is bound to the second molecule and the first molecule and the second molecule are in proximity. The hybridization product optionally is extended to form an extension product and the presence or absence of the hybridization product or extension product is identified, whereby the hybridization product or extension product is indicative of the first molecule and the second molecule being in proximity under the condition.

Also provided is a method for determining whether a molecule of interest is in association with a regulatory region in genomic DNA under a specific condition, which comprises inserting a nucleic acid comprising a homing sequence adjacent to the regulatory region in genomic DNA of a cell or inserting a nucleic acid comprising the regulatory region adjacent to a homing sequence into genomic DNA of a cell; exposing a sample comprising the cell to a condition; and contacting the sample with a first hybrid and a second hybrid, where the first hybrid comprises a first binding partner and a first nucleic acid and the second hybrid comprises a second binding partner and a second nucleic acid. The first binding partner specifically binds to the molecule of interest and the second binding partner binds to a molecule in association with the homing sequence. The first nucleic acid comprises a first nucleotide sequence complementary to a second nucleotide sequence in the second nucleic acid and is capable of forming a hybridization product with the second nucleic acid when the first hybrid is bound to the molecule of interest and the second hybrid is bound to the molecule in association with the homing sequence, and the molecule of interest and the molecule in association with the homing sequence are in proximity. The hybridization product optionally is extended to form an extension product and the presence or absence of the hybridization product or the extension product is identified, whereby the hybridization product or the extension product is indicative of the molecule of interest being in association with the nucleic acid regulatory sequence under the condition. The homing sequence sometimes is a lacO sequence and the molecule in association with the homing sequence sometimes is identical to or substantially identical to a lac repressor protein or a functional fragment thereof.

Provided also is a method for determining the nucleotide sequence of one or more fragments of genomic DNA in association with a molecule of interest under a specific condition, which comprises exposing a sample comprising a genomic DNA fragment in association with a molecule of interest to a specific condition and contacting the sample with a first hybrid and a second hybrid, where the first hybrid comprises a first binding partner and a first nucleic acid and the second hybrid comprises a second binding partner and a second nucleic acid. The first binding partner specifically binds to the molecule of interest and the second binding partner binds to double-stranded DNA or another molecule in association with the genomic DNA fragment. The first nucleic acid comprises a first nucleotide sequence complementary to a second nucleotide sequence in the second nucleic acid and is capable of forming a hybridization product with the second nucleic acid when the first hybrid is bound to the molecule of interest and the second hybrid is bound to double-stranded DNA or the other molecule in association with the genomic DNA fragment, and the molecule of interest and the genomic DNA or the other molecule are in proximity. The hybridization product optionally is extended to form an extension product and the hybridization product or the extension product and a genomic DNA fragment in contact with it are isolated. The nucleotide sequence of the genomic DNA fragment then is identified.

Many of the embodiments described herein are applicable to MAD assays in which a sample is exposed to a certain condition. The sample sometimes is a cell, a group of cells or is prepared from cells, and sometimes the sample is contacted by a cross-linking agent, for example. The sample can be exposed to any appropriate condition, including a condition that induces DNA damage (e.g., ionizing radiation) or induces mitogenic stimulation, and/or exposing the sample to an infective agent (e.g., a virus, bacterium, or fungus). The condition sometimes is exposure to an exogenous molecule or no exposure to an exogenous molecule. Any appropriate exogenous molecule can be added to the sample, including a nucleotide analog or derivative (e.g., bromodeoxyuridine (BrdU)) or a modified nucleotide (e.g., a biotinylated nucleotide)); a small organic or inorganic compound; an antisense nucleic acid (e.g., a PNA); a catalytic nucleic acid (e.g., a ribozyme); an inhibitory nucleic acid (e.g., a short inhibitory RNA (siRNA)); a polypeptide (e.g a cytokine or growth factor); an antibody or a peptide mimetic. Methods of making and using such exogenous molecules are known. For example, small organic or inorganic compounds often have a molecular weight of 10,000 g/mol or less, and sometimes have a molecular weight of 5,000 g/mol or less, 1,000 g/mol or less, or 500 g/mol or less. Compounds sometimes are obtained using combinatorial library methods, such as spatially addressable parallel solid phase or solution phase libraries; synthetic library methods requiring deconvolution; “one-bead one-compound” library methods; and synthetic library methods using affinity chromatography selection (e.g., DeWitt, et al., Proc. Natl. Acad. Sci. USA (1993) 90:6909; Erb, et al., Proc. Natl. Acad. Sci. USA (1994) 91:11422; Zuckermann, et al., J. Med. Chem. (1994) 37:2678; Cho, et al., Science (1993) 261:1303; Carrell, et al., Angew. Chem. Int. Ed. Engl. (1994) 33:2059; Carell, et al., Angew. Chem. Int. Ed. Engl. (1994) 33:2061; and Gallop, et al., J. Med. Chem. (1994) 37:1233). Methods for making and assessing ribozyme activity are known (see, e.g., U.S. Pat. Nos. 5,093,246; 4,987,071; and 5,116,742; Haselhoff & Gerlach, Nature (1988) 334:585-591 and Bartel & Szostak, Science (1993) 261:1411-1418), methods for designing and testing siRNA molecules are known (e.g., Elbashir et al., Methods 26:199-213 (2002) and http address www.dharmacon.com) and peptide mimetic libraries are described (see, e.g., Zuckermann, et al., J. Med. Chem. (1994) 37:2678-2685). MAD assays for detecting multiple antigens in a molecule

Provided herein are methods for detecting the presence of multiple antigens within a molecule (ie., intramolecular antigens). The methods generally are performed by contacting a sample with a hybrids that bind to molecular antigens in the same molecule. One embodiment is a method for detecting two or more binding regions in a target molecule, which comprises contacting the target molecule with a first hybrid, which comprises a first binding partner and a first nucleic acid, and a second hybrid, which comprises a second binding partner and a second nucleic acid. The first binding partner specifically binds to a first binding region of the target molecule and the second binding partner specifically binds to a second binding region of the target molecule. The first nucleic acid comprises a first nucleotide sequence complementary to a second nucleotide sequence in the second nucleic acid and is capable of forming a hybridization product with the second nucleic acid when the first hybrid is bound to the first binding region and the second hybrid is bound to the second binding region and the first binding region and the second binding region are in the target molecule. The presence or absence of the hybridization product is identified using any of the procedures described previously, whereby the presence of the hybridization product is indicative of the first binding region and the second binding region in proximity and in the target molecule.

Many of the embodiments described herein are applicable to methods for detecting binding regions in a molecule. In certain embodiments, the target molecule is a protein, the first binding region comprises amino acids in the protein and the second binding region comprises other amino acids in the protein. In other embodiments, the target molecule is a protein, the first binding region comprises amino acids in the protein and the second binding region comprises, consists of, or is a portion of a modification moiety linked to the protein, where the modification moiety sometimes is an ubiquitin moiety, a phosphoryl moiety, an alkyl moiety (e.g., methyl), an alkanoyl moiety (e.g., acetyl), an alkanoic acid or alkanoate moiety (e.g., a fatty acid) or a glycosyl moiety. In some embodiments, samples are prepared from Saos2 cancer cells and normal human fibroblast (NHF) cells and contacted with hybrids comprising antibody binding partners that specifically bind antigens in the p53 tumor suppressor. In certain embodiments, antibodies in the hybrids bind to the N-terminus (e.g., monoclonal antibody DO-1), the C-terminus (e.g., polyclonal antibody Ab421) or phosphoryl-serine 15 (e.g., monoclonal damage indicator antibodies (DIAs)) of p53. In other embodiments, antibodies binding to an acetylated, methylated or phosphorylated histone protein are detected. In some embodiments, the target molecule is a nucleic acid and the first binding region comprises or consists of one or more nucleotides and the second binding region is another region of the nucleic acid, such as a region comprising or consisting of a methyl moiety.

In certain embodiments, samples are exposed to different conditions or exogenous molecules, and molecular antigens detected by MAD assays as being in proximity with one another are compared between each condition or exogenous molecule. These embodiments are useful for determining which molecular antigens exist in a molecule when a sample is exposed to different conditions or exogenous molecules. For example, it can be determined whether a particular protein is post-translationally modified (e.g., phosphorylated, ubiquinated and/or acetylated) under various conditions. It also can be determined whether a nucleic acid is modified (e.g., methylated) under various conditions, which can be accomplished by reacting a sample with a hybrid that specifically binds to double-stranded DNA or BrdU-labeled DNA and another hybrid that specifically binds to methylated DNA.

MAD Assays for Detecting Multimerization of a Molecule

Featured herein is a method for identifying a multimer of a target molecule in a sample, which comprises contacting the sample with a first hybrid, which comprises a first binding partner and a first nucleic acid, and a second hybrid, which comprises a second binding partner and a second nucleic acid. The first binding partner is identical to the second binding partner and specifically binds to the target molecule, the first nucleic acid comprises a first nucleotide sequence complementary to a second nucleotide sequence in the second nucleic acid and is capable of forming a hybridization product with the second nucleic acid when the first hybrid and the second hybrid are bound to a multimer of the target molecule. The presence or absence of the hybridization product is identified, whereby the hybridization product is indicative of a multimer of the target molecule present in the sample. Control assays sometimes are performed with monomer molecules (e.g., a mutant form of a protein molecule that cannot multimerize), which are useful for ruling out false positive results or background signal levels.

Many of the embodiments described herein are applicable to methods for detecting multimerization of a molecule. In certain embodiments, samples are exposed to different conditions or exogenous molecules, and multimerization detected by MAD assays is compared between each condition or exogenous molecule. These embodiments are useful for determining whether multimerization occurs when a sample is exposed to different conditions or exogenous molecules.

MAD Diagnostics and Detection Systems

MAD assays and reagents can be used to detect a molecule or molecules in a wide variety of samples, including, but not limited to, clinical, industrial, agricultural and environmental samples. For example, sample material often is of medical, veterinary, environmental, nutritional or industrial significance, and include body fluids, such as urine, blood, serum, plasma, milk, sputum, fecal matter, lung aspirates, and exudates; microbial culture fluids; aerosols; crop materials; animal meat (e.g., for human consumption or animal feed); and soils and ground waters. Methods of obtaining such samples are known. A molecule or molecules from a variety of sources can be detected, including but not limited to, molecules in pathogens, viruses, bacteria, yeast, fungi, amoebae and insects; molecules in diseased or non-diseased pest animals such as mice and rats; molecules in diseased and non-diseased domestic animals, such as domestic equines, bovines, porcines, caprines, canines, felines, avians and fish; and molecules in diseased and non-diseased humans.

MAD hybrids sometimes are delivered to a sample from subject and sometimes are administered to a subject in vivo. In some embodiments, often in vivo embodiments, a targeting component that specifically binds to a hybridization product or extension product also is delivered. The hybridization product sometimes is extended by endogenous enzymes present in the sample or subject, and sometimes it is extended by exogenous components delivered to the sample or subject (e.g., a polymerase and/or nucleotides). In some embodiments, the targeting component comprises a detectable label and a nucleic acid binding agent that specifically binds to a nucleotide sequence in the hybridization product or extension product. The targeting component sometimes comprises a label that can be detected in vivo, such as a label capable of being detected by magnetic resonance imaging (MRI) and labels useful for nuclear medicinal applications. In embodiments where MAD hybrids are administered in vivo to a subject, binding partners from the same or similar organism as the subject (e.g., human or primate antibodies where the subject is human), or binding partners modified to include amino acid sequences from the subject (e.g., humanized antibodies where the subject is human), often are selected to reduce or prevent an immune response generated by the subject against one or more of the administered hybrids.

Diagnostics can be performed by contacting a sample with MAD hybrids that target different combinations of binding sites. In some embodiments, a MAD hybrid specifically binds to a cell-specific molecule and another MAD hybrid specifically binds to a disease-specific molecule. Such embodiments are useful for identifying whether a certain cell type in a sample carries a disease-specific marker, and are useful for determining progression of a disease or condition. Disease specific markers sometimes are molecules expressed by diseased cells, such as cancer cells, but not by non-diseased cells. A disease specific marker sometimes is a molecule expressed by a pathogenic organism but not the host organism invades, and sometimes is a molecule expressed by a cell invaded by a pathogenic organism and not by host cells not invaded by the organism. In some embodiments, a MAD hybrid specifically binds to a cancer-specific molecule, such as a marker specific for hepatocarcinoma cells, and one or more other MAD hybrids specifically bind to molecules expressed specifically by liver, colon, uterus, and kidney cells. Such embodiments are useful for determining cell types and organs that are diseased, and are useful for determining the extent to which a disease has spread. In related embodiments, a MAD hybrid that specifically binds to a molecule specific for a progressive stage of a disease can be included in the diagnostic, such as a hybrid that specifically binds to a molecule specific for metastatic cells but not non-metastatic cells.

A MAD hybrid is selected to specifically bind to a molecular marker specific to a cell type, diseased cell, or organism, and any marker specific to a cell type, diseased cell, or organism can be selected as a target for these diagnostic applications. Examples of specific markers include, but are not limited to, EBNAI a viral nuclear antigen found in EBV infected B-cells; S100P, S100A4, prostate stem cell antigen, lipocalin 2, claudins 3 and 4, and trefoil factors I and 2 in pancreatic adenocarcinoma (Int J Cancer. 2004 October 20;1 12(l):100-12); CD antigens, microphthalmia transcription factor (MITF), and members of the Bcl-2 family in neoplastic mast cells (Eur J Clin Invest. 2004 August;34 Suppl 2:41-52); a cell surface marker, such as CDs, HLAs, actins and tubulins as a healthy cell marker. Hybridization products or extension products can be detected using any of the methods described herein.

In certain embodiments, a MAD hybrid specifically binds to a molecule expressed by diseased cells and another MAD hybrid specifically binds to another molecule expressed by disease cells. Such embodiments are useful for diagnosing a disease in a subject where the disease is not specifically diagnosed by one marker but can be specifically diagnosed by detecting a combination of two or more markers in a sample. In these embodiments, one or more MAD hybrids that specifically bind to a cell type specific marker also can be utilized. The sample is from any source, and sometimes is a particular set of cells or group of cells from a subject.

In related embodiments, a MAD hybrid specifically binds to a molecule expressed by a particular organism and another MAD hybrid specifically binds to another molecule expressed by the organism. Similar to embodiments described above, these embodiments are useful for detecting an organism in a sample where the organism is not specifically detected by one marker but can be specifically detected by identifying two or more markers in a sample. In these embodiments, one or more MAD hybrids that specifically bind to a cell type specific marker also can be utilized. Such embodiments are useful for detecting a particular strain of organism in a sample (e.g., a biological sample, a sample from animal meat for human consumption, or an environmental sample), where the strain is specifically detected by a combination of a genus-associated molecule and a species-associated molecule, for example. The latter embodiments are useful for detecting a pathogenic organism in a biological sample for diagnosing a disease caused by the organism (e.g., hepatitis C infection in a human blood sample), and for detecting a particular organism in an environmental sample for agricultural and anti-bioterrorism applications. For example, MAD hybrids can be used to detect the presence, absence or levels of beneficial bacterial in soil to determine suitability for growing crops, and for detecting a pathogenic organism such as anthrax in soil or water samples for combating bioterrorism.

Thus, provided is a method for identifying a disease, condition, or organism from a sample, which comprises contacting a sample with a first hybrid and a second hybrid, where the first hybrid comprises a first binding partner and a first nucleic acid and the second hybrid comprises a second binding partner and a second nucleic acid. The first binding partner and second binding partner specifically bind to a first binding region or second binding region in a molecule, or a first molecule and a second molecule, where each molecule is independently selected from a molecule specifically expressed by a diseased cell, a molecule specifically expressed by a pathogenic organism, and a molecule specifically expressed by a certain cell type. The first nucleic acid comprises a first nucleotide sequence complementary to a second nucleotide sequence in the second nucleic acid and is capable of forming a hybridization product with the second nucleic acid when the first hybrid is bound to the first molecule or first molecular region and the second hybrid is bound to the second molecule or second molecular region, and the first molecule and the second molecule are in proximity or the first molecular region and second molecular region are in proximity. The hybridization product sometimes is extended by delivering a component that extends the hybridization product (e.g., a polymerase and/or nucleotides). The presence of absence of the hybridization product or extension product is identified, whereby the presence of the hybridization product or extension product detects the disease, condition or organism from the sample. In some embodiments, a targeting component that specifically binds to a nucleotide sequence in the hybridization product or extension product is delivered. The targeting component often comprises a detectable label, and sometimes the targeting component is detected by delivering a secondary agent that specifically binds to the targeting component and comprises a detectable label.

Also provided is a method for identifying a disease or condition in a subject, which comprises delivering a first hybrid and a second hybrid to a subject, where the first hybrid comprises a first binding partner and a first nucleic acid and the second hybrid comprises a second binding partner and a second nucleic acid. The first binding partner and second binding partner specifically bind to a first binding region or second binding region in a molecule, or a first molecule and a second molecule, where each molecule is independently selected from a molecule specifically expressed by a diseased cell, a molecule specifically expressed by a pathogenic organism, and a molecule specifically expressed by a certain cell type. The first nucleic acid comprises a first nucleotide sequence complementary to a second nucleotide sequence in the second nucleic acid and is capable of forming a hybridization product with the second nucleic acid when the first hybrid is bound to the first molecule or first molecular region and the second hybrid is bound to the second molecule or second molecular region, and the first molecule and the second molecule are in proximity or the first molecular region and second molecular region are in proximity. The hybridization product sometimes is extended by delivering exogenous components that extend the hybridization product (e.g., a polymerase and/or nucleotides), and a targeting component that specifically binds to a nucleotide sequence in the hybridization product or extension product is delivered. The targeting component often comprises a detectable label, and sometimes the targeting component is detected by delivering a secondary agent that specifically binds to the targeting component and comprises a detectable label. The hybrids, targeting component, and other diagnostic components are delivered in an amount effective to identify the disease or condition in the subject.

MAD Therapeutics

Many of the MAD hybrids disclosed herein for MAD diagnostics and detection systems are useful for therapeutic applications. In therapeutic embodiments, one or more MAD hybrids is delivered to an in vitro or ex vivo sample from a subject or administered to a subject in vivo. A hybridization product sometimes is extended by endogenous enzymes present in the sample or subject, and sometimes is extended by exogenous components delivered to the sample or subject (e.g., a polymerase and/or nucleotides).

The hybrids and hybrid nucleic acids sometimes are selected to yield a bioactive hybridization product or extension product after delivery or administration. In some embodiments, the hybridization product or extension product is internalized by cells to which the hybrids bind (e.g., one or more hybrids bind to a surface receptor internalized by a cell) and the internalized hybrid nucleic acids are transcribed within the cell (e.g., by RNA polymerase III) into short interfering RNA (siRNA) that inhibits expression of regulatory components in cells. The siRNA inhibition sometimes is short term (e.g., target RNA is inhibited) and may be long term (e.g., chromatin bearing the target gene is modified; see, e.g., Nature, 2004 August 15 online; Induction of DNA methylation and gene silencing by short interfering RNAs in human cells, Kawasaki H, Taira K). The nucleotide sequence in the hybridization product or extension product often is selected to include a RNA polymerase binding site, and the encoded siRNA sequence is selected to target any regulatory gene sequence or RNA sequence for a desired effect. For example, the encoded siRNA can inhibit expression of a factor that normally down-regulates cell apoptosis, and thereby can induce apoptosis and death in cells to which the MAD hybrids bind and form a hybridization product. In another example, the encoded siRNA can inhibit expression of an oncogene in cells to which the MAD hybrids bind and thereby treat cancer.

In some embodiments, a targeting component that specifically binds to a nucleotide sequence in a hybridization product or an extension product formed by the hybrids sometimes is delivered or administered. The targeting component sometimes is delivered or administered at the same time or after the MAD hybrid or hybrids are delivered or administered. Without being limited by theory, the targeting component is expected to specifically target cells to which MAD hybrids specifically bind and form a hybridization product or extension product, and not target cells without MAD hybridization products or extension products. The targeting component often comprises a nucleic acid binding agent that specifically binds to a nucleotide sequence in the hybridization product or extension product. Any nucleic acid binding agent can be utilized, including those described herein. The targeting component often comprises a cytotoxic agent, and in alternative embodiments, a secondary component that specifically binds to the targeting component comprises a cytotoxic agent is delivered to the sample or subject (e.g., an antibody that specifically binds to the targeting component and is linked to a cytotoxic agent). The cytotoxic agent sometimes directly kills cells, and includes, but is not limited to, a radioisotope, a chemotherapeutic, a toxin (e.g., ricin) and a molecule that induces apoptosis. The cytotoxic agent sometimes indirectly kills cells, such as by inducing an immune response that specifically kills cells bound to the targeting component (e.g., the targeting component comprises an HLA binding peptide or cytokine that elicits a CTL or HTL response). A MAD therapeutic processes sometimes is performed after a diagnostic test, such as a MAD diagnostic test described herein, is completed. Diseases and conditions include, but are not limited to, cell proliferation diseases and conditions (e.g., cancers and tumors) and pathogen-induced diseases and conditions. In some embodiments, the targeting component comprises a bioactive nucleic acid such as a dsDNA that encodes an siRNA, as described herein, and alternatively, a secondary component that binds to the targeting component comprises the bioactive nucleic acid.

MAD hybrids, targeting components and other agents useful for therapeutic applications (collectively referred to hereafter as “MAD therapeutic components”) are delivered to a subject in a suitable pharmaceutical formulation. Any pharmaceutically acceptable carrier can be formulated with the therapeutic components so long as the latter retains all or much of its therapeutic activity after administration. Examples of pharmaceutically acceptable carriers include but are not limited to a carrier, a diluent, an excipient, an auxiliary, a binder, a lubricant, a colorant, a disintegrant, a buffer, an isotonic agent, a preservative, an anesthetic, and the like which are used in a medical field. Pharmaceutical compositions comprising the peptide compositions may be manufactured by any known method, including but not limited to conventional mixing, dissolving, granulating, dragee-making, levigating, emulsifying, encapsulating, entrapping or lyophilizing processes.

A pharmaceutically acceptable carrier often is selected in part by the administration route for the composition. For example, routes of administration include but are not limited to topical administration, eye dropping, instillation, percutaneous administration, injection (e.g., subcutaneous, intracutaneous, intravenous, intraperitoneal), oral administration, inhalation, and the like. Also, the dosage form such as injectable preparations (e.g., solutions, suspensions, emulsions, solids to be dissolved), tablets, capsules, granules, powders, liquids, liposome inclusions, ointments, gels, washes, pads, patches, cosmetics, external powders, sprays, inhaling powders, eye drops, eye ointments, suppositories, pessaries, and the like often are selected in part on the administration method.

MAD therapeutic components generally are used in amounts effective to achieve the intended purpose of reducing the number of targeted cells; detectably eradicating targeted cells; treating, ameliorating, alleviating, lessening, and removing symptoms of the disease or condition; and preventing or lessening the probability of the disease or condition or reoccurrence of the disease or condition. A therapeutically effective amount sometimes is determined in part by MAD assays of samples from a subject, cells maintained in vitro, and experimental animals. For example, a dose can be formulated and tested in assays and experimental animals to determine an IC₅₀ value for killing cells. Such information can be used to more accurately determine useful doses.

Dosage amount and interval may be adjusted individually to provide MAD therapeutic component levels sufficient to maintain a therapeutic effect. Patient dosages for topical administration range from about 0.01 mg/day to about 100 mg/day. Patient dosages for administration by injection or oral administration range from about 0.1 to 5 mg/kg/day, often from about 0.5 to I mg/kg/day. Therapeutically effective levels may be achieved by administering multiple doses each day. The amount of MAD therapeutic components administered often is dependent on the subject being treated, the severity of the affliction, the manner of administration and the judgment of the prescribing physician. The dosage may vary within this range depending upon the dosage form employed and the route of administration utilized. The exact formulation, route of administration and dosage can be chosen by the individual physician in view of the patient's condition. (see e.g., Fingl et al., 1996, In: The Pharmacological Basis of Therapeutics, 9^{th} ed., Chapter 2, p. 29, Elliot M. Ross). The therapy may be repeated intermittently while symptoms are detectable or when they are not detectable. The therapy may be performed by administering the peptide composition in combination with one or more other agents that enhances the effectiveness of the composition for treating acne, examples of which are described above.

A therapeutically effective dose of the MAD therapeutic components will provide a therapeutic benefit without causing substantial toxicity. Toxicity of the peptide compositions described herein can be determined by standard pharmaceutical procedures in cell cultures or experimental animals, and assays described hereafter can be utilized to determine doses that yield a toxic effect. Sometimes, a therapeutically effective amount is guided by identifying a LD₅₀ value, which is the dose lethal to 50% of the population, or a LD₁₀₀, which is the dose lethal to 100% of the population. The dose ratio between toxic and therapeutic effect is the therapeutic index. MAD therapeutic compositions which exhibit high therapeutic indices often are utilized. The data obtained from cell culture assays and animal studies can be used to formulate a dosage range that is not significantly toxic for use in humans.

Thus, provided is a method for treating a disease or condition in a subject, which comprises delivering a first hybrid and a second hybrid to a subject or a sample from the subject, where the first hybrid comprises a first binding partner and a first nucleic acid and the second hybrid comprises a second binding partner and a second nucleic acid. The first binding partner and second binding partner specifically bind to a first binding region or second binding region in a molecule, or a first molecule and a second molecule, where each molecule is independently selected from the group consisting of a molecule specifically expressed by a diseased cell, a molecule specifically expressed by a pathogenic organism, and a molecule specifically expressed by a certain cell type. The first nucleic acid comprises a first nucleotide sequence complementary to a second nucleotide sequence in the second nucleic acid and is capable of forming a hybridization product with the second nucleic acid when the first hybrid is bound to the first molecule or first molecular region and the second hybrid is bound to the second molecule or second molecular region, and the first molecule and the second molecule are in proximity or the first molecular region and second molecular region are in proximity. The hybridization product sometimes is extended by delivering exogenous components that extend the hybridization product (e.g., a polymerase and/or nucleotides), and a targeting component that specifically binds to a nucleotide sequence in the hybridization product or extension product is delivered. The targeting component often comprises a cytotoxic agent, and sometimes a secondary agent is delivered that specifically binds the targeting agent and comprises a cytotoxic agent. The MAD therapeutic components (e.g., hybrids, targeting component, and secondary component) are delivered in an amount effective to treat or prevent the disease or condition in the subject.

MAD Assay Compositions and Kits

MAD assay compositions and kits are useful for performing MAD assays, diagnostics and therapeutics. For example, provided is a composition which comprises a nucleic acid derivitized at the 5′ end with a sulfhydryl- or amine-reactive chemical moiety (e.g., a maleimide or haloacetyl sulthydryl-reactive moiety or a isotriocyanate, succinyl ester or sulfonyl halide amine-reactive moiety) and a reagent for coupling the nucleic acid derivative to a binding partner (e.g., a reducing agent such as dithiothreitol (DTT) for coupling a haloacetyl- or maleimide-derivitized nucleic acid to an antibody or antibody fragment).

Also provided is a composition which comprises one or more hybrids capable of forming a hybridization product having an overlapping, partially double stranded region of six or fewer nucleotides in length. Provided also is a composition which comprises one or more hybrids in combination with a signal enhancer nucleic acid, examples of which are described herein. Also provided is a composition comprising three or more hybrids, where the one or the hybrids is capable of forming a hybridization product with two or more of the other hybrids. Provided also is a composition which comprises one or more hybrids in combination with a nucleic acid binding agent that specifically binds to a nucleotide sequence in a hybridization product formed by the hybrids or an extension product formed therefrom. The nucleic acid binding agent often comprises a detectable label (e.g., a fluorescent label, light emitting label, light scattering label, a radioisotope label); sometimes comprises a cytotoxic agent (e.g., ricin or a radioisotope); and sometimes comprises a solid support (e.g., sephadex or glass). The nucleic acid binding agent sometimes comprises a protein that specifically binds to a nucleotide sequence of the hybridization product or extension product formed by the hybrids, such as a lac repressor, a gal4 protein, a tus protein, a nucleic acid binding fragment of the foregoing and a nucleic acid binding sequence variant of the foregoing. In the foregoing compositions, the binding partner in one or more of the hybrids often is an antibody or an antibody fragment. Hybrids in the compositions sometimes are directed to different molecules or different binding regions in a molecule. The compositions sometimes include a sample, such as a sample disclosed herein.

Provided also is a composition which comprises one or more hybrids, where a binding partner of one or more of the hybrids is an antibody or antibody fragment, and the antibody or antibody fragment specifically binds to a particular antibody isotype (e.g., IgG₁ or IgG₂) or an antibody from a specific animal (e.g., a mouse or rat monoclonal antibody or a mouse, rat, rabbit, goat, hamster or chicken polyclonal antibody). Such compositions are useful in conjunction with primary binding agents that specifically bind to separate molecules or binding regions in a molecule. For example, one hybrid specifically binds to an IgG₁ primary antibody and another hybrid specifically binds to an IgG₂ primary antibody in certain embodiments. In other embodiments, one hybrid specifically binds to a mouse primary antibody and another hybrid specifically binds to a rat primary antibody. The compositions sometimes include a sample, such as a sample disclosed herein.

MAD kits comprise components of compositions described herein and often a set of instructions for using them to prepare one or more MAD reagents and/or to perform a MAD assay, diagnostic and/or therapeutic method. The instructions often describe one or more of the following: sample preparation procedures, conditions for contacting hybrids with the sample, extension conditions, detection conditions (e.g., PCR conditions), conditions for linking MAD assay components to a solid support, and a procedure for preparing a hybrid from binding partners and nucleic acids provided. In some embodiments, kits comprise a first container that includes a first hybrid comprising a first binding partner and a first nucleic acid, and a second container that includes a second hybrid comprising a second binding partner and a second nucleic acid. The first nucleic acid includes a first nucleotide sequence complementary to a second nucleotide sequence in the second nucleic acid and is capable of forming a hybridization product when the first hybrid and the second hybrid are in proximity. Kits sometimes include reagents for extending the hybridization product and reagents for detecting the extension product. The first hybrid and the second hybrid sometimes are primary binding agents that bind to a first molecule and a second molecule, respectively, and sometimes are secondary binding agents that bind to a first primary binding agent and a second primary binding agent, respectively. In certain embodiments the first primary binding agent is an antibody or antibody fragment from a first animal and the second primary binding agent is an antibody or antibody fragment from a second animal. In some embodiments, the first primary binding agent is an antibody or antibody fragment of a first isotype and the second primary binding agent is an antibody or antibody fragment of a second isotype. In particular embodiments, the first primary binding agent and the second primary binding agent are antibodies or antibody fragments from the same subtype of the same animal, and the hybrids recognize antibodies or antibody fragments of the subtype of the animal. Where MAD assay hybrids are utilized as secondary detection agents, kits sometimes include one or more primary binding partners (e.g., antibodies or antibody fragments) to which the hybrids specifically bind.

Kits sometimes include oligonucleotides and nucleotides for amplifying hybrid nucleic acids, hybridization products or extension products by an amplification process such as PCR. In some embodiments, kits include oligonucleotides useful for amplifying unique multiplexing identification sequences in hybridization products and extension products. In an embodiment, the kit includes reagents for preparing hybrids from antibodies and nucleic acids provided by the user or provided in the kit. In some embodiments, the kits comprise a cross-linking agent (e.g., formaldehyde) for performing in situ MAD assays or MAD-ChIP assays. Kits sometimes include a solid support for immobilizing MAD assay components (e.g., microscope slide or biotin-, streptavidin- or avidin-derivitized solid support) and sometimes include modified nucleotides (e.g., biotin-modified nucleotides) for generating extension products or amplification products that can be immobilized to a solid support. In some embodiments, kits include a nucleic acid binding agent linked to a detectable label (e.g., lac repressor protein linked to fluorescein) for use as a reporter molecule in in situ MAD assays, for example. In certain embodiments, a kit comprises an agent for introducing an exogenous nucleotide sequence to cellular genomic DNA (e.g., a recombinase-mediated cassette exchange plasmid). In some embodiments, the kit includes an enzyme or cells useful for performing a MAD assay, such as Klenow polymerase, Taq polymerase, enzymes for performing TMA, and/or a transfection competent cell line.

The following examples illustrate but do not limit the invention.

### EXAMPLE 1

### Oligonucleotide Design and Generation of MAD-PCR Reagents

Hybrid nucleic acids are designed to include several features: the nucleotide sequences are not significantly homologous to mammalian genomic DNA likely to be tested; the oligonucleotides do not form internal hairpin structures; a hybrid nucleic acid linked to one binding partner forms a greater number of complementary interactions with the hybrid nucleic acid to which it hybridizes as compared to the hybrid nucleic acids to which it is not designed to hybridize; the nucleotide sequence is optimized for routine PCR analysis, TaqMan® primer probe design and LUX™ probe design; and the nucleic acid includes desired targets, markers or flags. For the hybrid nucleic acids discussed in this Example, a lac repressor binding site is divided between the two overlapping sequences formed in the hybridization product such that neither nucleic acid includes a complete lac repressor binding site. The lac repressor binding determinant is approximately 22 base pairs (bp) and the overlap region of the hybridization product represents only 5 base pairs (bp) or 9 bp of the binding determinant. The starting sequence for the lac repressor binding determinant was from E. coil embedded in the backbone sequence from Bombyx mori (silkworm) genomic DNA. Several computer programs were used to achieve the parameters listed above: Oligo 4.0 (National Biosciences Inc.), Primer Express (Applied Biosystems, Inc.) and LUX™ web-based primer design (www.invitrogen.com).

Specific hybrids were generated as follows. The oligonucleotide 5′-CTGTGTAGATAGTTTACTGCCCTGAACCCTGATCGAAAGACCTGGTCACAAATTGTTATCCG CTC-3′ was synthesized having a 5′ PEG spacer and terminated by a 5′ amino moiety (synthesized by Trilink, Inc., using #PEG-282). The oligonucleotide was synthesized with a phosphodiester backbone and the 3′ end was unmodified. This oligonucleotide was reacted at a molar ratio of about 5:1 (oligo:Ab) with a monoclonal antibody that specifically binds to human p53 (DO-I, anti-aa2l-25, Oncogene Research Products Cat #OP43), where the antibody was previously modified with succinimidyl 6-hydrazinonicotinate acetone hydrazone (SANH) at a ratio of 15:1 (SANH:Ab). The resulting hybrid was purified by size exclusion chromatography, stored at 4° C. and designated “DO-1” hybrid. [01281 Similarly, a SANH-activated monoclonal antibody that specifically binds to the V5 epitope tag (Invitrogen Cat #R960) was linked to one of two oligonucleotides: 5′-amino-PEG-ACACAGCAGACTGAGTTTGATCCATTAACGAACCTTCACGATACCTGACGACTTGTGGAATT GTGAGCG-3′ or 5′-amino-PEG-ACACAGCAGACTGAGTTTGATCCATTAACGAACCTTCACGATACCTGACGACTTGTGGAATT GTGAGCGGATAA-3′. These hybrids were referred to as “anti-V5-5” hybrid and “anti-V5-9” hybrid, respectively, according to the length of the overlap in the hybridization product generated in combination with the DO-1 hybrid.

### EXAMPLE 2

### MAD-PCR Assay

This example describes a method in which two target molecules are detected using two unique hybrids. The physiologic response of many mammalian cells to insults that cause DNA damage is stabilization of p53 protein expression levels. A consequence of this stabilization is transcriptional activation of the HDM2 gene by p53. The HDM2 protein will bind p53 and target it for eventual degradation. p53/HDM2 binding is enhanced in cells treated with proteosome inhibitors such as epoxomicin and MG-132 (“EM” treatment; Calbiochem Cat #'s 324800 and 474790). In this example, hybrids are designed and utilized to specifically detect p53/MDM2 complexes in a sample.

Mouse 2KO cells deleted for p53 and MDM2, the latter of which is the murine equivalent of HDM2 (Montes et al., Nature 378: 203-206 (1995)), were transiently transfected with human p53 and V5-tagged HDM2-expressing plasmids at gene ratios that yield a comparable protein signal according to a Western procedure following EM treatment. p53 and V5-tagged HDM2-expressing plasmids were generated by extracting from human cDNA the p53 gene (GenBank Accession No. NM_{—}000546, version NM_{—}000546.2 GI:8400737) and HDM2 gene (GenBank Deposit No. NM_{—}002392, Version NM_{—}002392.1 GI:4505136) by PCR using the primers

5′-GGGGACAAGTTTGTACAAAAAAGCAGGCACCATGGAGGAGCCGCAG TCAG-3′
and
5′-GGGGACCACTTTGTACAAGAAAGCTGGGTTGCGGTCTGAGTCAGG CCCTTCTGT-3′ for p53,
and
5′-GGGGACAAGTTTGTACAAAAAAGCAGGCACCATG-TGCAATACCAACATG-3′
and
5′-GGGGACCACTTTGTACAAGAAAGCTGG-GTTGCGGGGGAAATAAGTTAGC-3′ for HDM2.

The resulting PCR products were suitable for cloning into a recombinase mediated subcloning system (Gateway™ system, Invitrogen Corporation). PCR products first were recombined into pDONR221 plasmid and sequenced. Subsequently, the open reading frame for each gene was moved into pcDNA3.2-DEST for expression in the 2KO cells.

Cell lysates are prepared in lysis buffer (50 mM Tris 8.0,5mM EDTA, 150 mM NaCl, 0.5% NP-40 supplemented with a protease inhibitor cocktail) and maintained at 4° C. The lysates are subjected to centrifugation at 14,000×g for 10 minutes at 4° C. and the supernatant is transferred to a new tube. The hybrid combinations DO-1 and anti-V5-5 or DO-1 and anti-V5-9 are added at final concentrations of about 1 nM to 100 nM each (often 10 nM to 20 nM) and incubated overnight at 4° C. with gentle rocking. Lysates contacted with single hybrids also are assembled as controls. The next day, each mixture (i.e., lysate with hybrid(s)) is diluted such that hybrid concentrations are equal to or less than 20 μM, and 12 μl of diluted mixture is added to a 30 μl volume total extension reaction containing 4 mM each deoxynucleotide, 10 mM MgCl₂ and 1 unit of Sequenase™ (USB Corp). Incubation is at 4° C. for 20 minutes.

2 μl of each extension reaction is used in a 50 μl PCR reaction containing primers directed against the hybrid nucleic acid sequences distal to the overlap region, which are referred to as “detection primers.” Detection primers have the sequence 5′-CCCTGAACCCTGATCGAAAG-3′ and 5′-TTAACGAACCTTCACGATACCTGA-3′. PCR reactions, 30 cycles with a 42° C. annealing temperature are run on 11% acrylamide gels and amplified products are revealed by ethidium bromide (EtBr) staining. The expected product is 86 bp. The amplification product is amenable to TaqMan® RT-PCR quantification in conjunction with a double labeled probe oligonucleotide: 5′-FAM™-TGGTCACAAATTGTTATCCGCTCACAATTCC-TAMRA™-3′ (available from Applied Biosystems, Inc.).

### EXAMPLE 3

### MAD Quantification Assay

This example describes a method in which one target molecule is detected with two pools of related hybrids. Mouse 2KO cells, deleted for both p53 and MDM2, are transiently transfected with a human p53 expressing plasmid at a range of gene doses (see Example 2). A human cell line containing p53 at an unknown concentration and the same cell line subjected to a genotoxic stress (e.g. ionizing radiation or UV radiation) also are prepared.

Lysates are prepared and are contacted with hybrids and hybridization products are extended and amplified by PCR as described in Example 2. In this method, however, the two hybrids are formed from two aliquots of the same anti-human p53 polyclonal antibody. The two aliquots are conjugated, separately, to two different activated oligonucleotides which can form an overlapped dsDNA structure, such as the oligonucleotides described in Example 1. Each PCR signal for the control dose curve and the unknown samples is quantified by gel separation, EtBr staining and densitometry or by an RT-PCR method.

### EXAMPLE 4

### MAD Multiplexing Assay

This example describes methods in which several target molecules are detected in a sample using several unique hybrids. Mouse 2KO cells, deleted for both p53 and MDM2, are transiently transfected with a human p53, HDM2 or human p300 expressing plasmids singly or in combination at a range of gene doses. A test human cell line containing p53, HDM2 and p300 is treated with or without genotoxic stresses.

Lysates are prepared and are contacted with hybrids and hybridization products are extended and amplified by PCR as described in Example 2. The hybrids, DO-1, anti-HDM2 and anti-p300, each are composed of a monoclonal antibody with the specificity indicated. The oligonucleotide conjugated to each monoclonal antibody has the following characteristics: the DO-I oligonucleotide has a unique PCR priming site and an overhang as shown for DO-I in Example 1. The anti-HDM2 oligonucleotide is identical to the anti-V5 oligonucleotide described in Example 1, where it has a unique PCR priming site and its overhang is complementary to that of DO-I. The anti-p300 oligonucleotide has the same overhang as the oligonucleotide in the anti-HDM2 hybrid, such that it too can form a duplex with the DO-1 hybrid, but its unique PCR priming site is different than the other two and is spaced differently than the HDM2 oligonucleotide such that it generates a product of different length than the anti-HDM2 hybrid, (i.e., 96 bp) when it forms a hybridization product with the DO-1 hybrid. The oligonucleotide linked to the anti-p300 antibody has the sequence 5′-PEG-ACGTATGCTATGCTATGAGTATGAACACAGAAGACTGAGTTAGATCGACGACTTGTGGAAT TGTGAGCG-3′.

PCR is accomplished using three detection primers singly and in pairs. The two detection primers listed above yield an 86 bp product when the p53/HDM2 proteins are in proximity and the hybrids form a hybridization product. A third primer having the sequence 5′-TATGAGTATGAACACAGAAGACTGAGTTAGA-3′, is specific for the anti-p300 hybrid and yields a product only when a hybridization product forms between the anti-p300 hybrid nucleic acid and the anti-p53 hybrid nucleic acid. The PCR signal of the control dose curve and the unknown samples is quantified by gel separation, EtBr staining and densitometry or by an RT-PCR method. Both primer pairs are amenable to TaqMan® RT-PCR quantification in conjunction with a double-labeled probe oligonucleotide: 5′-FAM™-TGGTCACAAA-TTGTTATCCGCTCACAATTCC-TAMRA™-3′.

### EXAMPLE 5

### In Situ MAD Assay

This example describes a method in which two complexed target molecules are detected using two unique hybrids in fixed cells. A hybridization product is developed using a lac repressor reporter molecule. Cells containing target molecules of interest are grown on glass cover slips and optionally are treated with exogenous agents. Cells are fixed and permeablized using formaldehyde, acetic acid or methanol. Typically, the fixation method is consistent with a method optimized for the antibody in a standard immunofluorescence assay (IFA). Hybrids are contacted in PBS or TBS with or without detergent. The cover glasses are rinsed in buffer without hybrids. A brief extension reaction is performed in a Tris-based buffer with MgCl₂ and deoxynucleotides, and the reaction is terminated by repeated rinsing. The extended hybrids are detected by the addition of a biotinylated lac repressor at a low concentration. Lac repressor is commercially available and is readily biotinylated using the same SANH procedure described in Example 1. The unbound lac repressor is rinsed away and the location of the bound lac repressor is revealed by development with commercially available FITC-conjugated avidin and epifluorescent microscopy. Additional fluorescent signal amplification steps are added as necessary. Texas red-phalloidin and DAPI are used as counterstains for subcellular organelles.

### EXAMPLE 6

### Intramolecular MAD assay

This example describes a method in which two epitopes on a single target molecule are detected using two unique hybrids. Mouse 2KO cells, deleted for both p53 and MDM2, are transiently transfected with a human p53 expressing plasmid at a range of gene doses (see Example 2). A human cell line containing p53 at an unknown concentration and the same cell line exposed to a genotoxic stress also are prepared.

Lysates are prepared and are contacted with hybrids, and hybridization products are extended and PCR amplified as described in Example 2. The two hybrids utilized are an anti-human p53 monoclonal antibody and a monoclonal antibody that specifically binds to a variable p53 epitope such as an antibody that specifically binds to human p53 phosphoryl-serinel 5 moiety, ubiquitin moiety or SUMO moiety (small ubiquitin-related modifier (Mahajan et al., Cell 88: 97-107 (1997)). The two monoclonal antibodies are conjugated separately to two different activated oligonucleotides which can form an overlapped hybridization product (see Example 1 and Example 4). Each PCR signal for the control dose curve and the unknown samples is quantified by gel separation, EtBr staining and densitometry or by a RT-PCR method.

This method is adapted to a solid phase by resolving samples using polyacrylamide gel electrophoresis (PAGE) and transferring sample components from the gel to an appropriate membrane. The membrane is contacted with hybrids formulated in a blocking buffer (e.g., BLOTTO) and unbound hybrids are removed by rinsing the membrane. Hybridization products formed between hybrids are extended in a Tris-based buffer with MgCI₂ and deoxynucleotides and the reaction is terminated by repeated rinsing. The extended hybrids are detected by the addition of a low concentration of a biotinylated lac repressor (see Example 5). The unbound lac repressor is rinsed away and the location of the bound lac repressor is revealed by development with a horse radish peroxidase (HRP)-conjugated anti-biotin antibody and commercial chemiluminescent cocktails.

### EXAMPLE 7

### MAD Multimerization Assay

This example describes a method in which one target molecule is detected with two unique but related hybrids. Mouse 2KO cells, deleted for both p53 and MDM2, are transiently transfected with a human p53-expressing plasmid at a range of gene doses. Putative multimerization mutants of p53 are transiently expressed in parallel.

Lysates are prepared and are contacted with hybrids and hybridization products are extended and PCR amplified as described in Example 2. Hybrids are formed from two aliquots of the same anti-human p53 monoclonal antibody. The monoclonal antibody can bind only one epitope per molecule, and accordingly, a hybridization product is indicative of two molecules being in contact with one another, i.e., multimerized. The two aliquots are conjugated, separately, to two different activated oligonucleotides which can form a hybridization product when bound to antigens in close proximity (e.g., see Example 1 and Example 4). Each PCR signal for the control dose curve and the unknown samples is quantified by gel separation, EtBr staining and densitometry or by a RT-PCR method.

### EXAMPLE 8

### MAD-ChIP Assay

This example describes a method in which two chromatin-associated target molecules are detected using two unique hybrids. Cells containing molecules of interest are grown and optionally treated with exogenous agents. Cells are fixed with formaldehyde and harvested (see e.g., Kuo & Alis, Methods 19(3):425-433 (1999)). The chromatin is fragmented by a suitable fragmentation method such as sonication, and optionally, separated from free protein and naked DNA by density gradient centrifugation (see, e.g., Solomon, et al., Cell 17:937-947 (1997)) and buffer is exchanged. Hybrids directed against chromatin associated proteins, a DNA bound homing protein, or modified DNA nucleoside incorporated prior to harvest are contacted and extended either in pairs or are multiplexed (see Example 4). The mixture is passed over a lac repressor affinity column to recover linked hybrids and their cargo. Bound material is released from the column by a pH, salt or detergent gradient or pulse. Nucleic acid within the eluted complexes is recovered by heating to reverse the formaldehyde crosslinks, or by proteinase K treatment to degrade the antibodies and cellular proteins followed by organic extraction and ethanol precipitation. The DNA is separated into a sub-lSObp component (duplex MAD-oligos) and a greater than 150bp component (e.g., genomic DNA from complexes retained by the lac repressor column) by a method such as electrophoresis, size exclusion chromatography or rate-zonal centrifugation. The greater than 150bp DNA is used as a target for PCR analysis as in traditional ChIP. Alternatively, the eluate is treated with heat to reverse the formaldehyde crosslinks and the constituent proteins resolved by electrophoresis, detected by a method such as silver staining, or identified by method such as MALDI-TOF mass spectrometry.

The hybrids are generated from a polyclonal serum (e.g., Example 3) or from monoclonal antibodies (e.g., Example 2 or Example 4). Regardless of the type of antibodies utilized to generate the hybrids, purified DNAs are used to construct a library by exposing the DNA mixture to Taq polymerase and then TA-cloning using TOPO-TA (Invitrogen Corporation) reagents and methods.

Each document and publication cited herein is incorporated by reference in its entirety, including all figures, tables, drawings, and documents cited therein.

1. A method for detecting a first molecule and a second molecule in a sample, which comprises:
contacting a sample with a first hybrid, which comprises a first binding partner and a first nucleic acid, and a second hybrid, which comprises a second binding partner and a second nucleic acid, wherein:
the first binding partner specifically binds to the first molecule and the second binding partner specifically binds to the second molecule,
the first nucleic acid comprises a first nucleotide sequence complementary to a second nucleotide sequence in the second nucleic acid and is capable of forming a hybridization product with the second nucleic acid when the first hybrid is bound to the first molecule and the second hybrid is bound to the second molecule and the first molecule and the second molecule are in proximity;
diluting the sample and the hybrids, wherein the molecules and hybrids are not in association with a solid phase;
extending the hybridization product to form an extension product after diluting the sample and the hybrids; and
identifying the presence or absence of the extension product, whereby identifying the extension product detects the first molecule and the second molecule in the sample.

2. The method of claim 1, wherein the first molecule and second molecule are independently selected from the group consisting of a non-hybrid nucleic acid, a protein, a protein in association with another protein, and a protein in association with a non-hybrid nucleic acid.

3. The method of claim 1, which comprises contacting the sample with a cross-linking agent before contacting the sample with the hybrids.

4. The method of claim 1, wherein the first nucleotide sequence and second nucleotide sequence are six or fewer nucleotides in length.

5. The method of claim 1, which comprise contacting the system with a third nucleic acid, wherein the third nucleic acid hybridizes to a single-stranded region in the first nucleic acid or second nucleic acid in the hybridization product and abuts the 3′ end of the other of the first nucleic acid or second nucleic acid when hybridized.

6. The method of claim 5, wherein there is no gap between the 5′ end of the third nucleic acid and the 3′ end of the first nucleic acid or second nucleic acid.

7. The method of claim 1, wherein the first binding partner, second binding partner, or first binding partner and second binding partner is an antibody or antibody fragment.

8. The method of claim 1, wherein the presence or absence of the extension product is identified by contacting the system with polymerase chain reaction (PCR) reagents and detecting the presence or absence of PCR products.

9. A method for detecting a first molecule and a second molecule in a sample, which comprises:
contacting a sample with a first hybrid, which comprises a first binding partner and a first nucleic acid, and a second hybrid, which comprises a second binding partner and a second nucleic acid, wherein:
the first binding partner specifically binds to the first molecule and the second binding partner specifically binds to the second molecule,
the first nucleic acid comprises a first nucleotide sequence complementary to a second nucleotide sequence in the second nucleic acid and is capable of forming a hybridization product with the second nucleic acid when the first hybrid is bound to the first molecule and the second hybrid is bound to the second molecule and the first molecule and the second molecule are in proximity;
optionally extending the hybridization product to form an extension product;
contacting the hybridization product or extension product with a nucleic acid binding agent that specifically binds to a nucleotide sequence in the hybridization product or extension product; and
identifying the presence or absence of the nucleic acid binding agent bound to the hybridization product or extension product, whereby identifying the nucleic acid binding agent bound to the hybridization product or extension product detects the first molecule and the second molecule in the sample.

10. The method of claim 9, wherein the nucleic acid binding agent comprises a nucleic acid binding protein or a fragment thereof that specifically binds to a nucleotide sequence in the hybridization product or extension product.

11. The method of claim 10, wherein the nucleic acid binding agent comprises a lac repressor protein, a gal4 protein, a tus protein, or a fragment thereof.

12. The method of claim 9, wherein the nucleic acid binding agent comprises a detectable label.

13. The method of claim 9, wherein one or more of the hybrids are in association with a solid phase.

14. A composition which comprises a first hybrid, which comprises a first binding partner and a first nucleic acid, and a second hybrid, which comprises a second binding partner and a second nucleic acid, wherein:
the first nucleic acid comprises a first nucleotide sequence complementary to a second nucleotide sequence in the second nucleic acid and is capable of forming a hybridization product with the second nucleic acid; and
the first nucleotide sequence and second nucleotide sequence are six or fewer nucleotides in length.

15. The composition of claim 14, wherein the first binding partner, the second binding partner, or the first binding partner and second binding partner are an antibody or antibody fragment.

16. A composition which comprises:
a first hybrid, which comprises a first binding partner and a first nucleic acid, and a second hybrid, which comprises a second binding partner and a second nucleic acid, wherein the first nucleic acid comprises a first nucleotide sequence complementary to a second nucleotide sequence in the second nucleic acid and is capable of forming a hybridization product with the second nucleic acid; and
a third nucleic acid capable of hybridizing to a single-stranded region of the first nucleic acid or second nucleic acid in the hybridization product, wherein the 5′ end of the third nucleic acid abuts the 3′end of the other of the first nucleic acid or second nucleic acid when hybridized.

17. The composition of claim 16, wherein the first binding partner, the second binding partner, or the first binding partner and second binding partner are an antibody or antibody fragment.

18. A composition which comprises:
a first hybrid, which comprises a first binding partner and a first nucleic acid, and a second hybrid, which comprises a second binding partner and a second nucleic acid, wherein the first nucleic acid comprises a first nucleotide sequence complementary to a second nucleotide sequence in the second nucleic acid and is capable of forming a hybridization product with the second nucleic acid; and
a nucleic acid binding agent that specifically binds to a nucleotide sequence in the hybridization product or a nucleotide sequence in an extension product produced by extending the hybridization product.

19. The composition of claim 18, wherein the first binding partner, the second binding partner, or the first binding partner and second binding partner are an antibody or antibody fragment.

20. The composition of claim 18, wherein the nucleic acid binding agent comprises a lac repressor protein, a gal4 protein, a tus protein, or a fragment thereof that specifically binds to a nucleotide sequence in the hybridization product or extension product.

21. The composition of claim 18, wherein the nucleic acid binding agent comprises one or more components selected from the group consisting of a detectable label, a cytotoxic agent and a solid phase.

22. A method for detecting a molecule in a sample, which comprises:
contacting a sample with a first hybrid, which comprises a first binding partner and a first nucleic acid, and a second hybrid, which comprises a second binding partner and a second nucleic acid, wherein:
the first binding partner specifically binds to a first binding region in the molecule and the second binding partner specifically binds to a second binding region in the molecule,
the first nucleic acid comprises a first nucleotide sequence complementary to a second nucleotide sequence in the second nucleic acid and is capable of forming a hybridization product with the second nucleic acid when the first hybrid is bound to the first binding region and the second hybrid is bound to the second binding region;
diluting the sample and the hybrids, wherein the molecule and hybrids are not in association with a solid phase;
extending the hybridization product to form an extension product after diluting the sample and hybrids; and
identifying the presence or absence of the extension product, whereby identifying the extension product detects the molecule in the sample.

23. A method for detecting a molecule in a sample, which comprises:
contacting a sample with a first hybrid, which comprises a first binding partner and a first nucleic acid, and a second hybrid, which comprises a second binding partner and a second nucleic acid, wherein:
the first binding partner specifically binds to a first binding region in the molecule and the second binding partner specifically binds to a second binding region in the molecule,
the first nucleic acid comprises a first nucleotide sequence complementary to a second nucleotide sequence in the second nucleic acid and is capable of forming a hybridization product with the second nucleic acid when the first hybrid is bound to the first binding region and the second hybrid is bound to the second binding region;
optionally extending the hybridization product to form an extension product;
contacting the hybridization product or extension product with a nucleic acid binding agent that specifically binds to the nucleotide sequence of the hybridization product or extension product; and
identifying the presence or absence of the nucleic acid binding agent bound to the hybridization product or extension product, whereby identifying the nucleic acid binding agent bound to the hybridization product or extension product detects the molecule in the sample.
